(19) United States
(12) Patent Application Publication
(10) Pub. No.: US 2025/0006349 A1
(43) Pub. Date: Jan. 2, 2025

(54) SYSTEM AND METHOD FOR POPULATION BASED, REMOTE THERAPEUTIC MONITORING LEVERAGING MULTI-TIERED PUBLISH AND SUBSCRIBE ARCHITECTURE

(71) Applicant: Lifeguard Health Networks, Inc., Radnor, PA (US)

(72) Inventors: Martin Carty, Wayne, PA (US); Stephen Malaby, Devon, PA (US)

(21) Appl. No.: 18/606,121

(22) Filed: Mar. 15, 2024

Related U.S. Application Data
(60) Provisional application No. 63/524,691, filed on Jul. 2, 2023.

(51) Int. Cl. G16H 40/20; G06Q 40/08

(52) U.S. Cl. CPC G16H 40/20; G06Q 40/08

(57) ABSTRACT

Therapeutic monitoring systems and their use by participants are disclosed herein. The participants include without limitation patients, patient representatives, health care providers, health care payers, research institutions and other types of stake holders in the healthcare world. More particularly, the disclosed systems and methods relate to the creation, use and amendment of policy templates containing regimens, where a system of creation, use and amendment of the templates allows for practically unlimited tiers of participants and the non-conflicting movement of both templates and regimen customization through the disclosed system.

### CROSS-REFERENCE TO RELATED APPLICATIONS

This application claims priority to U.S. Provisional Application Ser. No. 63/524,691, filed Jul. 2, 2023, the contents of which are incorporated herein in their entirety by reference. This application is also filed concurrently with U.S. Provisional Appl. No. ______ and entitled “System and Method for Protecting Patients' Therapeutic Treatment Context Used for Training Artificial Intelligence Systems” (Atty. Dkt. No. 266-0004PUS); U.S. Provisional Appl. No. ______ and entitled “System and Method for Therapeutic Event Management and Value Attribution (Proof of Value) in Decentralized Health Information Network” (Atty. Dkt. No. 266-0006PUS); and U.S. Provisional Appl. No. ______ and entitled “System and Method for AI Assisted Therapeutic Micro-Interventions In Care at Home” (Atty. Dkt. No. 266-0007PUS), each of which is incorporated herein by reference in its entirety.

### TECHNICAL FIELD

This disclosure relates generally to therapeutic monitoring and treatment of medical patients primarily outside of a hospital setting. More particularly, the disclosure relates to a system and method for a multi-tier publish/subscribe framework, which includes version control and population change management, to support remote therapeutic monitoring at scale.

### BACKGROUND

U.S. Publication No. US2014/0207486 to Carty et al. and assigned to Lifeguard Health Networks, Inc. is hereby incorporated by reference in its entirety as background information and to aid in the understanding of more advanced concepts discussed herein.

Both research studies and anecdotal reports agree that at-home medical treatment and recovery may be safer, cheaper, and more effective than traditional hospital care, but only if the patients are closely monitored. The benefits of home care may be especially important for patients who are vulnerable to either infections that are common in hospitals, or other complications of inpatient care. In addition, some studies indicate that well monitored healthcare at home may be more effective at improving patient outcomes than traditional hospitalization. Furthermore, most experts agree that home healthcare can save money for both patients and payer organizations, including governments.

Healthcare outside of a hospital or other dedicated medical environment benefits from fulsome patient surveillance because surveillance allows healthcare professionals to more granularly evaluate patient progress as well as patient adherence and response to therapies. Not surprisingly, there is a medical surveillance marketplace offering labor, techniques, and technology to monitor patients' health and safety when those patients are away from medical facilities. The technology employed in marketable solutions may include hardware such as sensors, cameras, and other devices that can track a patient's vitals, wellness, movements, or other activities. There is also software at either or both of the system or client application level, which can help a patient report on subjective matters and help enforce the proper administration of therapies. In all, these technologies provide healthcare professionals with real-time or near-real-time information about a patient's condition and other medically relevant activities, which allows more accurate and timely medical interventions. Sample companies and products that participate in this marketplace are as follows: Health Hero; Lifeguard Health Networks (the assignee); Health Buddy; CARDIONET; WellDoc; FirstAlert; OnStar; Alora; AxisCare; Gyant; Medopad; Chronisense Medical; Ejenta; Cardiomo; 100 Plus; Vitls; Neteera; ContinUse Biometrics; iHeath; Binah.ai; MatrixCare; ContinuLink; ShiftCare; CareSmartz360; AdaCare; CareVoyant; AxxessHome; 10Aexchange; HealthSnap; CadenceCare; Health Recovery Solutions; Accuhealth; and TimeDoc Health.

Certain solutions for remote patient monitoring are static, hard coded data models that require universal updates across all parties and global adherence to a single definition. This monolithic approach limits best practice logic and/or customization that may be added by users (e.g., providers or other participants in the healthcare marketplace) with more relevant knowledge regarding the needs of patient cohorts or individual patients. By hindering the flexibility of downstream healthcare providers, researchers and administrators, the static and hard coded models lock all participants into the same data model universally. That result may hinder differentiation of remote surveillance services, may limit the availability of more customized patient-centric care, and may potentially burden the clinical teams with manual application of customizations.

### SUMMARY

Embodiments of the disclosure occur in a context of a healthcare ecosystem, where there are patients, family, healthcare providers, insurers, and many other types of medical-related entities. Many embodiments relate to surveillance of patients, primarily by healthcare providers and family, but sometimes insurers and other medical related entities in a contextually relevant sense and without violating confidentiality rights. Many embodiments relate to a system that employs a network, endpoint devices and software to connect a patient with all the relevant healthcare providers and medical-related entities for addressing the patient's healthcare related activity. In some embodiments, one mechanism for connecting all the networked participants is a policy template, which may be a collection of therapeutic regimens related to patient's medical condition or diagnoses. The regimens relate to medically relevant issues such as symptoms, vitals, medication, wellness, and procedures. Furthermore, according to certain embodiments, a policy template that embodies these regimens may be created and edited for particular diagnoses and ultimately for particular patients. The creation and editing of these policy templates occur in the network of certain embodiments and, through the actions of participants performing the following functions: creating a policy template, publishing a policy template, subscribing to a policy template (e.g., that was previously published), customizing or editing a policy template, prescribing a policy template to a patient, and using a policy template by a patient.

Some embodiments of the disclosure are directed toward supporting clinical best practices for remote therapeutic monitoring of patient cohorts across populations at scale (e.g., millions of patients) while enabling customization and personalization of policy templates by medical-related entities subscribing to those templates or patients having those templates prescribed to them. When, for example, a clinical best practice is updated/improved/changed at the publisher level (e.g., by a publisher of a policy template), the downstream subscribed entities, sub entities, and their prescribed patients may have their respective copies of the policy template seamlessly updated, while simultaneously protecting any and all customizations performed on a template by subscribers at their respective levels, i.e., without “overwriting” data customized by the owner of a particular policy template copy.

In order to service the proposed system, in some embodiments, a logical client-server approach is maintained where the server maintains a data repository (e.g., one or more databases) and server application software is used to service requests and interactions with all clients. The physical implementation of this arrangement may be in any conventional or known computing network. One example may be a logical server implemented with one or more physical computers that may or may not be geographically dispersed. The logical server may implement database software to store policy templates as well as metadata and information to track subscribers, publishers, policy template activity, and related activity in the network. For example, some embodiments of the present disclosure may use database software to associate each policy template with one or more database records, where the database records retain information including metadata, template versions, subscribers to templates or template versions, publishers of templates or versions of templates, authorizations for access, edit activity of templates, etc.

In more specific embodiments, the disclosed subject matter supports a unique multi-tiered (N-Tier) publish/subscribe model for remote therapeutic monitoring policy templates leveraged by third-party administrators of health (e.g., Federal Ministry(s) of Health, US insurers, health plans, etc.) to support institutional partners (e.g., health systems, hospital networks, community health systems, general practitioners, etc.), their affiliate organizations and partners (e.g., regional entities, practices, etc.) and ultimately to managed patient populations. For certain embodiments, when published/subscribed therapeutic policy templates are revised/enhanced (e.g., versioned), the embodiments of the system automatically update the therapeutic monitoring content across all “subscribed” organizational entities across all ecosystem and network system tiers, including any prescribed patient cohort populations, and without “overwriting” any customization/personalization completed by the entity subscribed to that therapeutic template. The multi-tiered publish and subscribe framework provides confidence to subscribing entities that their customizations to the therapeutic templates and/or unique parameters or safety thresholds set and assigned to their patient cohort will persist with high fidelity.

The system's multi-tiered publish/subscribe framework for remote therapeutic policy templates enables enterprise version control across all policy-based elements of its therapeutic policy templates including therapeutic side effects and symptoms toxicity, vital measures (biotelemetry), medication management, elements of wellness, procedures, as well as behavioral health surveys. The system has change management functionality that enables a seamless and automated change management at scale (millions of prescribed patients) for subscribing organizations and patients alike.

The foregoing summary is not intended to summarize each potential embodiment or every aspect of the present disclosure.

### BRIEF DESCRIPTION OF THE DRAWINGS

For illustration, there are shown in the drawings certain examples described in the present disclosure. In the drawings, like numerals indicate like elements throughout. The full scope of the subject matter disclosed herein is not limited to the precise arrangements, dimensions, and instruments shown. In the drawings:

FIG. 1 shows an illustrative network and hardware context for some embodiments.

FIG. 2 shows an exemplary tiered arrangement of participants and potential template communications paths.

FIGS. 3A, 3B and 3C show exemplary functions, communication paths, and processes related to templates.

FIGS. 4A and 4B show exemplary embodiments including processes and network and hardware relationships with data and tiering.

FIGS. 5A and 5B show exemplary logic and processes for template creation.

FIG. 6 shows exemplary logic and processes for editing/customizing symptom regimens.

FIG. 7 shows exemplary logic and processes for editing/customizing vitals regimens and/or wellness regimens.

FIG. 8 shows exemplary logic and processes for editing/customizing medication regimens.

FIG. 9 shows exemplary logic and processes for editing/customizing procedures regimens.

FIG. 10 shows exemplary logic and processes for editing/customizing goals regimens.

FIG. 11 shows exemplary logic and processes for editing/customizing file/attachment regimens.

FIGS. 12 and 13 show exemplary logic and processes for reconciling customizations.

FIG. 14 is a block diagram of an exemplary computer.

FIG. 15 schematically illustrates components of network nodes for a distributed network system according to the present disclosure.

### DETAILED DESCRIPTION

Embodiments of the present disclosure propose methods, systems, and software to improve aspects of remote patient monitoring technology. Policy-based therapeutic templates are employed to facilitate multiple aspects of therapeutic treatment, surveillance, and intervention. While the number of therapeutic aspects treated in a single policy template are not limited, many embodiments employ at least Seven general aspects: symptom surveillance; vitals; medication; wellness; procedures; files/attachments; and goals.

In the following description, for purposes of explanation, numerous specific details are set forth in order to provide a thorough understanding of the disclosed concepts. As part of this description, some of this disclosure's drawings represent structures and devices in block diagram form in order to avoid obscuring the novel aspects of the disclosed concepts. In the interest of clarity, not all features of an actual implementation are described. Moreover, the language used in this disclosure has been principally selected for readability and instructional purposes and may not have been selected to precisely delineate or circumscribe the claimed subject matter, leaving resorting to the claims as potentially the most expedient way to determine such claimed inventive subject matter. Reference in this disclosure to “one embodiment” or to “an embodiment” or “embodiments” means that a particular feature, structure, or characteristic described in connection with the embodiment is included in at least one embodiment of the disclosed subject matter, and multiple references to “one embodiment,” “an embodiment,” “some embodiments,” “many embodiments,” etc. should not be understood as necessarily all referring to the same embodiment.

It will be appreciated that in the development of any actual implementation (as in any software and/or hardware development project), numerous decisions must be made to achieve the developers' specific goals (e.g., compliance with system- and business-related constraints), and that these goals may vary from one implementation to another. It will also be appreciated that such development efforts might be complex and time-consuming, but would nonetheless be a routine undertaking for those having the benefit of this disclosure and being of ordinary skill in art.

1. Generally, symptom surveillance may refer to symptoms/side effects/flare ups/behavioral health events as experienced by a patient and in some embodiments, may be characterized by one or more questions to the patient regarding symptoms. In those embodiments, questions may be answered in any conventional manner, including without limitation in free form written or voice entry, by selecting from a menu, or by a combination of both techniques.

2. Also generally, and applying to certain embodiments, vitals may refer to measured physiologic characteristics, typically reflected with the help of a device. For example, blood pressure, pulse rate, body temperature, heart rate, blood oxygen levels, blood glucose, respiration rate, and weight may be considered examples of vitals.

3. Medication refers to therapeutic medicines in the colloquial sense, such as aspirin, Lipitor, Crestor, Advil, Motrin, etc.

4. Wellness may refer to observable characteristics such as nutrition, exercise, hydration, bowel movements, mood, and sleep typically reflected by a subjective measure.

5. Procedures refer to treatment processes having one or more therapeutic steps, such as changing a bandage, replacing a catheter, or collecting a sample.

6. Files/attachments refers to electronic items generally related to the subject therapies, which may include any file type or reference such as audio and/or video media, documents, references to external electronic sources, or references to external real sources. Some example files/attachments include tutorials, directions, information related to medications, treatments, symptoms, illnesses, side effects, medical care teams or team members, care facilities, equipment, mental health, clinical trials, or any information related to the subject therapy or therapeutic template.

7. Finally, goals generally refer to desired outcomes for the patient or therapy, such as a therapeutic status, physical or mental ability, tolerance to medication or stimuli, or other outcome related to the subject therapy or therapeutic template.

Some embodiments provide for sharing policy templates with any number of other network participants. Many embodiments also facilitate tracking the customizations/edits and movements of policy templates as they are propagated through a network of participants, which may include patients, patient representatives, healthcare providers, insurers, and other medically related entities. Even though policy templates may be shared with any number of participants at any level of either the healthcare or network ecosystems, embodiments of the disclosure are able to propagate policy template updates from publishers at any level to subscribers at any other level. Furthermore, embodiments provide that customizations made by a subscriber on its own copy of a template need not be altered by the presence of conflicting data in an update.

### A. Physical Context

Embodiments described herein disclose a network enabled (e.g., TCIP/IP enabled) continuity of care network including one or more computing devices, sensors and systems for communications and data sharing. These networks enable people on a caregiver team to receive and send medically relevant communications with a patient or patient representative in response to health events, incidents, questions, observations, trends, and the like. A continuity of care network may be doctor initiated and payer or insurer approved and is part of one possible network embodiment of this disclosure. In many embodiments, patients are invited onto an embodiment of the system by a healthcare provider or medical-related entity with a relationship to the patient's care. As healthcare often involves a support system around the patient, embodiments allow patients to invite family, friends, and other health advocates to join the system as a patient representative. One or more computing devices for each system participant may be joined into the network/system to run or access software related to the embodiments herein and access associated functionality.

Many embodiments propose technology and solutions through a collaborative care network, including devices having software made available for use by various participants in the healthcare ecosystem and marketplace. For example, depending upon the embodiment, software delivering all or part of the solution may reside on mobile devices, computers, thin clients, or intelligent devices of any kind that are commonly used by patients, patient representatives, healthcare professionals, insurers, government, medically related entities such as institutions, health systems, research entities, and all manner of healthcare provider organizations. As implemented, the software may have multiple general application areas. Healthcare providers and other healthcare related entities (such as insurers and institutes) may use a version of software (e.g., application) adapted for endpoint operating systems such as Microsoft Windows or Mac OS, or server operating systems (e.g., for S2S solutions) such as Linux or Windows Server. In addition, some embodiments allow providers and other entities access to software functionality through (i) mobile applications (e.g., IOS, Android, Java); (ii) a web portal accessed through a web browser on any device; or (iii) any information access and/or delivery system such as virtual or augmented reality tools. In some embodiments, these options may be facilitated through known client-server protocols using computer servers in a data center, public cloud, or other known computing resources.

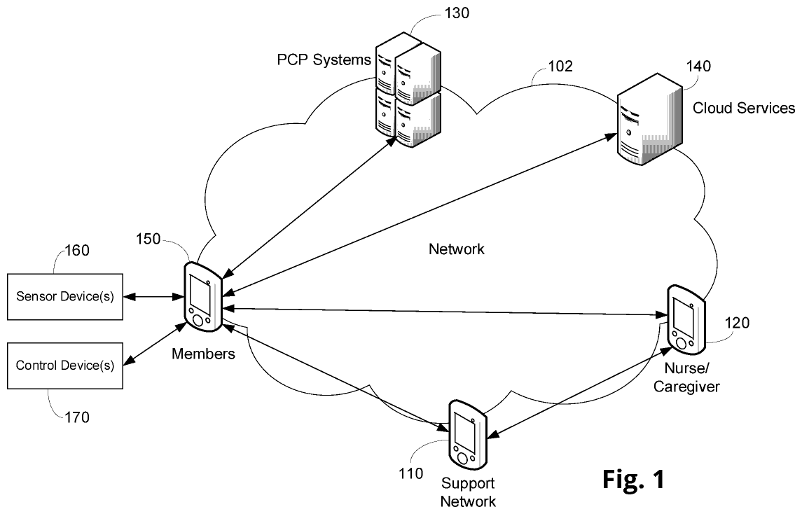

FIG. 1 shows an exemplary continuity of care network system 100. As shown in FIG. 1, the network system 100 is decentralized with computing power, data capture and monitoring and other functionality distributed and available at each of the various components. Patients or their representatives typically interface with/connect to the network system 100 with patient devices 150. Patient device 150 may be an edge-of-network computing device (e.g., a computer or mobile device such as a smartphone or tablet), optionally configured to monitor patient data based upon a specified health service policy. For example, a device may monitor the patient's biotelemetry, location, or other items depending upon the capability of the device. The patient's device 150 may also receive inputs from other devices such as sensors 160 or control devices 170. Patient device 150 may transmit patient data across one or more networks 102 to other devices and systems in the network system 100, such as one or more representative devices 110, a nurse or other caregiver device 120, one or more primary care provider systems 130, and servers/databases such as cloud services systems 140. Of course, network system 100 may include any number of additional devices and systems or may omit one or more devices or systems shown in FIG. 1.

Patient representative devices 110 may be used by those appointed by or on behalf of the patient, such as trusted family, friends, and other caregivers of a patient to assist with daily therapy compliance, program adherence, communications, health management, and the like. Network system 100 may include multiple patient representative devices 110 coupled to (i.e., in communication with) a patient device 150, thereby providing a few-to-one or many-to-one support network for the patient. By including multiple people's devices with customized privacy and intervention alert levels within the network, the responsibility of monitoring patient data may be distributed amongst a patient's family or other care representatives or network participants. Additionally, such a group may provide monitoring redundancy, for example if one representative does not have access (e.g., internet access) others in the patient's support network may monitor patient's data. In addition, the aspects of the network system 100 associated with a patient may be dynamic, expanding and contracting in response to a patient's needs, schedule, and the like.

Representative network devices 110 and nurse/caregiver device 120 may be any type of computing devices, such as any computing endpoint or mobile computing device, thereby enabling monitoring the patient substantially all times. Devices on the network system 100 may receive substantially real-time health data from patient device 150 to allow for a timely and effective intervention mechanisms.

Network system 100 may also include one or more healthcare provider systems 130 (e.g., PCP—or primary care provider systems). These systems 130 may periodically receive patient data from patient device 150, optionally as relayed through one or more servers. Medical practitioners may use systems 130 to diagnose patients, monitor effectiveness of therapy regimens, access EHR systems and facilitate similar functions or other tasks related to the provision of healthcare. Network system 100 may allow medical providers to monitor and diagnose many patients while simultaneously allowing designated family, friends, nurses, and the like to also monitor the patient's therapy regimen compliance, potential emergency and other situations or statuses.

Network system 100 may include or access cloud resources 140. Cloud resources 140 may provide a broad range of services, including using autonomous agents (for example, bots) for monitoring patient data received from patient device 150 and, at times, responding to patient data by sending a message or control signal to patient device 150 or contacting escalation services (e.g., “911”) according to rules defined by the applicable health service policy. Autonomous agents may provide continuous (i.e., 24/7) monitoring and may trigger appropriate responses in response to any type of event (these responses could include providing a message to a patient in response to a missed dose, contacting patients in response to a trending health incident, or contacting emergency services in the event of a detected emergency, for example). Cloud resources 140 may also include databases, dynamic resource registries, health service policies, monitoring services, data analytics, on-boarding and patient activation services, and the like.

A patient device 150 may also contain health service policy logic and algorithms configured to continuously monitor the patient's health events. In certain embodiments, local health service policy logic may trigger appropriate responses to any type of event.

Network system 100 may also illustrate a functional and communication relationship between many computing devices. However, each computing device's configurations may be governed by a patient-centered policy templates discussed below. In many embodiments, the patient-centric policy template provides a functional hierarchy and structure to the relationship of the computing devices making up network system 100, including providing escalation and intervention services and payer approval/preapproval services.

The devices and systems comprising network system 100 may be operatively coupled by one or more networks 102. Network 102 may include one or more of the Internet, local area networks (“LANs”), wide area networks (“WANs”), mobile telecommunications networks (e.g., 3G and 4G networks), and any suitable type of communication mediums. As illustrated in FIG. 1, the network system 100 may include one or more decentralized computing/communication devices (110, 120, 130, 140, 150) configured to communicate with one or more other computing devices. For example, each of a plurality of computing devices may use mobile health client software executed on a computing device using a microprocessor, random access and fixed memory, input, and output devices, and facilitating common communication protocols such as Bluetooth, Wi-Fi and 4G and/or 5G data and voice access. Some embodiments may capture and store patient data in a distributed fashion, for example, on the patient's computing device, rather than storing and accessing data on a central server or plurality of distributed servers.

Each communication device (110, 120, 150) in the decentralized network 102 may originate communications to any other communication device in the decentralized network. Thus, the decentralized network may allow for scaling of compliance and intervention tasks. For example, upon detection of a compliance event, the patient device 150 or a connected device/server may determine if the detected compliance event should be escalated to an incident such that a notification or alert should be sent to a health professional or patient representative. Thus, embodiments may allow for a direct one-to-many communication notifying any of the system participants of a compliance event or other event or status. The embodiments also allow for many-to-one communications, for example, from health care providers or patient representatives to the patient. Certain embodiments may also provide for direct communication between the patient communication device 150 and intervention services if the there is a determination that that level of escalation is desired, for example a healthcare professional or even 911.

In addition to the patient side solutions for the patient and his/her representatives, some embodiments of the disclosure may employ a decentralized patient network operations console (e.g., 120 in FIG. 1) for healthcare providers, healthcare professionals, nurses, social workers, etc. as well as for non-professionals responsible for managing multiple patients. A console/dashboard may be dynamic and driven by functional role, permissions and availability of resource enabling real-time monitoring, intervention, and mobilization of resources in response to any health event/status/escalation. This type of console functionality may enable a healthcare professional (physician, nurse, nurse practitioner, nutritionist, an allied health professional, and/or a health coach) to have a personalized dashboard specific to their role and expertise so they may provide continuity of care services concurrently to a number of members each with their own discrete, individually tailored physician directed parameters of therapeutic care.

### i. Sensors

Embodiments contemplate the use of sensors that determine vitals, wellness, or other health-relevant information about a patient. Examples of these types of sensors are thermometers, scales, blood oxygen sensors, blood pressure cuffs, and similar devices that provide telemetry/biotelemetry readings or any therapeutically relevant status or reading related to a patient. The patient's computing device may operate as a sensor or may receive data from the sensors in real-time, near real-time, periodically, or event-based, when a sensor reading or measured trend passes a threshold, etc. Certain embodiments may use standard (e.g., cross-platform (e.g., iOS, Android, BlackBerry OS, Windows Phone 7, etc.)) plug-ins to interact with devices. Additionally, embodiments may be deployed using existing or to-be-developed computing systems and communication platforms.

### ii. Network Operation

Medical-related organizations may be onboarded to embodiments of the system either by invitation from the system administrator or by enrolling in a program associated with the system. Patients may be onboarded in any manner for onboarding individuals to software resources. For example, they may be onboarded to the network system 100 and become network participants using a link or invitation provided, for example, by a healthcare provider that already is a participant. A patient representative may be similarly onboarded or alternatively receive a link or invite from the patient and facilitated by the patient's mobile application. Patient representatives may be designated with one or more particular roles, thereby defining their capabilities in the system.

### iii. Patient Data

Embodiments of the disclosure contemplate the handling of patient data consistent with government regulations (e.g., HIPPA) and patient privacy expectations. In most embodiments, all patient health information (PHI) is protected by both access restrictions and encryption. For certain embodiments, the access limitations are determined by the patient's permissions and/or by an associated healthcare provider. Encryption or other data protection techniques are employed relative to best practices and expected to evolve over time.

In some embodiments, all data in the system is maintained in one logical location, such as a database, designated or agreed by the system administrator. Alternatively, more sensitive data may be maintained closer to and in storage controlled by the data owner (e.g., healthcare provider or patient), with less sensitive data flowing to a centralized logical database or server.

Varying embodiments of the disclosure provide for access to data being designated based upon one or more of a per datum basis, a per patient basis, a per provider basis, a per publisher basis, a per subscriber basis or otherwise. In one or more embodiments, multiple entities may have access to the patient data or portions thereof. For example, for a certain patient policy template, the patient data may be shared with multiple providers engaged in the treatment of the patient; perhaps a primary care physician, a medical oncologist, a surgeon, and a physical therapist. In some embodiments, entities that are not providers may also have access to the data or portions thereof, such as insurers, researchers, system developers, health networks, institutions, or organizations.

### B. Policy Templates

Embodiments of the disclosure employ policy templates for implementing the therapeutic surveillance discussed herein. In one or more embodiments, a policy template is constructed as a grouping of object oriented or object-based regimens. Conceptually, embodiments of the present disclosure are not limited in the number of regimens in a template, so there may be as many regimens as the authoring organization requires or desires to adequately manage a therapeutic condition at scale. In many embodiments, a policy template is created and deployed around a diagnosis or group of (e.g., related) diagnoses, and it provides a surveillance specification relative to those diagnoses or post operative procedures. The regimens may be general in nature, but specific in implementation. For example, the regimens for many embodiments are patient symptoms, vitals, medication, wellness, procedures, files/attachments, and goals. While regimens are discussed further below, generally, each regimen may designate schedules, compliance rules, and alert and/or notification thresholds. The following examples provide illustration regarding these regimens and the aspects.

### i. Sample Policy Template Regimens

The following tables show sample regimens to aid in the understanding of the disclosure. By way of example, the samples help illustrate nuances (e.g., menus and selections) that are difficult to show in the logic and process diagrams. A skilled artisan will appreciate that application of the samples to the diagrams and understand how details in the samples represent expanded possibilities in the logic flows.

With respect to the samples, highlighting (e.g., bold text) is used to show issue or menu selection that is illustrated by any particular table or table portion.

### ii. Symptom Regimen Examples

| | Chronic Obstructive Pulmonary | |
|---|---|---|
| Therapeutic Monitoring | Disease (COPD)/Pleural | Chemo Systemic Therapy |
| Template | Effusion with Catheter | (Thoracic Cancer) |
| Symptom assessment | COPD Symptoms - Daily | Chemo Symptoms - Weekly |
| (grouping) regimen # 1 | | |
| Name(s) of Symptoms | Shortness of Breath | Constipation |
| in Assessment | Dizziness | Cough |
| (grouping) #1 | Numbness & Tingling | Decreased Appetite |
| | General Pain | Diarrhea |
| | Fatigue | Fatigue |
| | Nail Discoloration | General Pain |
| | | Heartburn |
| | | Hot Flashes |
| | | Numbness & tingling |
| | | Painful Urination |
| | | Shortness of Breath |
| | | Vomiting |
| Symptom threshold | Example: Shortness of Breath | Example: General Pain |
| question(s) | Severity - In the last ‘x’ | Occurrence: In the last ‘x’ |
| | (hours/days), what was the | (hours/days), how OFTEN did |
| | SEVERITY of your SHORTNESS | you have GENERAL PAIN? |
| | OF BREATH at its WORST? | Severity - In the last ‘x’ |
| | Occurrence: In the last ‘x’ | (hours/days), what was the |
| | (hours/days), how OFTEN did | SEVERITY of your GENERAL |
| | you have SHORTNESS OF BREATH? | PAIN at its WORST? |
| | Level of Interference - In the | Level of Interference - In the |
| | last ‘x’ (hours/days), how did | last ‘x’ (hours/days), how did |
| | SHORTNESS OF BREATH | GENERAL PAIN INTERFERE |
| | INTERFERE with your usual or | with your usual or daily |
| | daily activities? | activities? |
| | Yes or No | Yes or No |
| | Or Custom question | Or Custom question |
| Each Symptom | Never | Never |
| Threshold Alert Logic | Greater or equal | Greater or equal |
| (Rules Based Alerts) | Less or equal | Less or equal |
| | Equal | Equal |
| Each Symptom | Primary Value: Severity | Primary Value: Occurrence |
| Question has Threshold | None | Never |
| Rule VALUES | Mild | Rarely |
| | Moderate | Occasionally |
| | Severe | Frequently |
| | Very Severe | Almost Constantly |
| | Occurrence | Severity |
| | Never | None |
| | Rarely | Mild |
| | Occasionally | Moderate |
| | Frequently | Severe |
| | Almost Constantly | Very Severe |
| | Interference | Interference |
| | Not at all | Not at all |
| | A little bit | A little bit |
| | Somewhat | Somewhat |
| | Quite a bit | Quite a bit |
| | Very much | Very much |
| | Custom | Custom |
| Each Symptom has a | Alert if primary measure (is | Alert if primary measure (is |
| secondary “Trending” | “greater than”) for more than | “greater than”) for more than |
| rule based upon the | “#” consecutive measures. | “#” consecutive measures. |
| PRIMARY question | | |
| (Severity, Occurrence, | | |
| Interference) | | |
| Each “scheduled” | Are you concerned about this | Are you concerned about this |
| symptom has a level of | measure? (yes) or (no) | measure? (yes) or (no) |
| concern question (if | Answer: Yes, escalates | Answer: Yes, escalates |
| outside of acceptable | Symptom measure to | Symptom measure to |
| threshold) | provider of record. | provider of record. |
| | Answer: No, adds measure to | Answer: No, adds measure to |
| | charts but does not escalate | charts but does not escalate |
| | to provider of record. | to provider of record. |
| Symptom Assessment | Daily | Daily |
| Schedule - Frequency | Weekly | Weekly (Friday) |
| | Monthly | Monthly |
| | Custom Days | Custom Days |
| | Custom Weekly Days | Custom Weekly Days |
| Symptom Assessment | Morning, Noon, Afternoon, | Morning, Noon, Afternoon, |
| Schedule - Time of Day | Evening, Bedtime | Evening, Bedtime |
| | Specific Time of Day | Specific Time of Day |
| | As Needed | As Needed |
| Symptom Assessment | Alert if compliance is _{——}_{——}_{——}% | Alert if compliance is _{——}_{——}_{——}% |
| Compliance rules | over _{——}_{——}_{——} consecutive days. | over _{——}_{——}_{——} consecutive days. |
| Symptom assessment | Daily Inspection of Catheter | N/A |
| (grouping) regimen # 2 | | |
| Name(s) of Symptoms | Signs of Infection at Catheter | N/A |
| in Assessment | Site | |
| (grouping) #2 | (custom) | |
| | Volume of Fluids Discharged | |
| | from Catheter (custom) | |
| | Color of Fluids Discharged | |
| | from Catheter (custom) | |
| Symptom threshold | In the last 24 hours, have you | N/A |
| question(s) | experienced tenderness, | |
| | swelling, or infection at the | |
| | catheter site? | |
| | In the past 24 hours, what | |
| | was the volume of Fluids | |
| | discharged from your | |
| | Catheter? | |
| | In the last 24 hours, what | |
| | was the color of the fluids | |
| | discharged from your | |
| | Catheter? | |
| Each Symptom | Never | N/A |
| Threshold Alert Logic | Greater or equal | |
| (Rules Based Alerts) | Less or equal | |
| | Equal | |
| Each Symptom | None, mild, moderate, severe, | N/A |
| Question has Threshold | very severe | |
| Rule VALUES | 0-5 ml | |
| | 6-10 ml | |
| | 11-20 ml | |
| | 21-30 ml | |
| | Over 30 ml | |
| | Clear, Cloudy, Yellow, Pink, | |
| | Red/Brown | |
| Each Symptom has a | Alert if primary measure (is | N/A |
| secondary “Trending” | _{——}_{——}_{——}) for more than “x” | |
| rule based upon the | consecutive measures. | |
| PRIMARY question | | |
| (Severity, Occurrence, | | |
| Interference) | | |
| Each “scheduled” | Are you concerned about this | N/A |
| symptom has a level of | measure? (yes) or (no) | |
| concern question (if | Answer: Yes, escalates | |
| outside of acceptable | Symptom measure to provider | |
| threshold) | of record. | |
| | Answer: No, adds measure to | |
| | charts but does not escalate | |
| | to provider of record. | |
| Symptom Assessment | Daily | N/A |
| Schedule - Frequency | Weekly | |
| | Monthly | |
| | Custom Days | |
| | Custom Weekly Days | |
| Symptom Assessment | Morning, Noon, Afternoon, | N/A |
| Schedule - Time of Day | Evening, Bedtime | |
| | Specific Time of Day | |
| | As Needed | |
| Symptom Assessment | Alert if compliance is _{——}_{——}_{——}% | N/A |
| Compliance rules | over _{——}_{——}_{——} consecutive days. | |

### iii. Medication Regimen Examples

| | Chronic Obstructive Pulmonary | |
|---|---|---|
| Therapeutic Monitoring | Disease (COPD)/Pleural | Chemo Systemic Therapy |
| Template | Effusion with Catheter | (Thoracic Cancer) |
| Medication Regimen | Penicillin | Tamoxifen |
| Medication Type | Pill | Pill |
| | Strip | Strip |
| | Injectable | Injectable |
| | Custom | Custom |
| Dosage | 250 mg | 10 mg |
| | custom | custom |
| Quantity | Number (1) | Number (1) |
| Medication Regimen | Daily | Daily |
| Schedule - Frequency | Weekly | Weekly (Friday) |
| | Monthly | Monthly |
| | Custom Days | Custom Days |
| | Custom Weekly Days | Custom Weekly Days |
| | As Directed | As Directed |
| Medication Regimen | Morning, Noon, Afternoon, | Morning, Noon, Afternoon, |
| Schedule - Time of Day | Evening, Bedtime (Two times) | Evening, Bedtime (One Time) |
| | Specific Time of Day | Specific Time of Day |
| | As Directed | As Directed |
| Compliance Rule | Alert if compliance is | Alert if compliance is |
| | less than % over _{——}_{——}_{——} | lessthan % over _{——}_{——}_{——} |
| | consecutive days | consecutive days |

### iv. Vital Regimen Examples

| | Chronic Obstructive Pulmonary | |
|---|---|---|
| Therapeutic Monitoring | Disease (COPD)/Pleural | Chemo Systemic Therapy |
| Template | Effusion with Catheter | (Thoracic Cancer) |
| Vital Regimen | Glucose | Glucose |
| | Blood Pressure | Blood Pressure |
| | Weight | Weight |
| | Pulse Oximetry | Pulse Oximetry |
| | Body Temperature | Body Temperature |
| | Heart Rate (Resting) | Heart Rate (Resting) |
| | Respiratory Rate | Respiratory Rate |
| Units | Percentage % | beats per minute |
| Acceptable Range | 92-100 | 60-100 |
| Machine Entry Required | Yes or No | Yes or No |
| (Data provenance) | | |
| Schedule (Frequency) | As Needed | As Needed |
| | Daily | Daily |
| | Weekly | Weekly |
| | Custom Days | Custom Days |
| | Custom Weekly Days | Custom Weekly Days |
| Schedule (Time of Day) | Morning, Noon, Afternoon, | Morning, Noon, Afternoon, |
| | Evening, Bedtime | Evening, Bedtime |
| | Specific Time of Day | Specific Time of Day |
| | As Directed | As Directed |
| Trending Rule(s) | Compliance: | Compliance: |
| | Alert if compliance is less | Alert if compliance is less |
| | than _{——}_{——}_{——}% over _{——}_{——}_{——} | than _{——}_{——}_{——}% over _{——}_{——}_{——} |
| | consecutive days | consecutive days |
| | Range: | Range: |
| | Alert if measure is less than | Alert if measure is less than |
| | _{——}_{——}_{——}% out of acceptable range | _{——}_{——}_{——}% out of acceptable range |
| | for more than _{——}_{——}_{——}# of | for more than _{——}_{——}_{——}# of |
| | consecutive recordings | consecutive recordings |
| | Average Range: | Average Range: |
| | Alert if daily average vital | Alert if daily average vital |
| | measure is less than _{——}_{——}_{——}% | measure is less than _{——}_{——}_{——}% |
| | out of acceptable range for | out of acceptable range for |
| | more than _{——}_{——}_{——}# of | more than _{——}_{——}_{——}# of |
| | consecutive days | consecutive days |
| | Measure Out of Range: | Measure Out of Range: |
| | Alert if measure is outside of | Alert if measure is outside of |
| | acceptable range _X_{—} | acceptable range _X_{—} |
| Vital Regimen | Glucose | Glucose |
| | Blood Pressure | Blood Pressure |
| | Weight | Weight |
| | Pulse Oximetry | Pulse Oximetry |
| | Body Temperature | Body Temperature |
| | Heart Rate | Heart Rate |
| | Respiration Rate | Respiratory Rate |
| Units | Breaths Per Minute (BPM) | Fahrenheit (F.) |
| | | Celsius © |
| Acceptable Range | 12-20 | 96-99.9 |
| Schedule (Frequency) | As Needed | As Needed |
| | Daily | Daily |
| | Weekly | Weekly |
| | Custom Days | Custom Days |
| | Custom Weekly Days | Custom Weekly Days |
| Schedule (Time of Day) | Morning, Noon, Afternoon, | Morning, Noon, Afternoon, |
| | Evening, Bedtime | Evening, Bedtime |
| | Specific Time of Day | Specific Time of Day |
| | As Directed | As Directed |
| Trending Rule(s) | Compliance: | Compliance: |
| | Alert if compliance is less | Alert if compliance is less |
| | than _{——}_{——}_{——}% over _{——}_{——}_{——} | than _{——}_{——}_{——}% over _{——}_{——}_{——} |
| | consecutive days | consecutive days |
| | Range: | Range: |
| | Alert if measure is less than | Alert if measure is less than |
| | _{——}_{——}_{——}% out of acceptable range | _{——}_{——}_{——}% out of acceptable range |
| | for more than _{——}_{——}_{——}# of | for more than _{——}_{——}_{——}# of |
| | consecutive recordings | consecutive recordings |
| | Average Range: | Average Range: |
| | Alert if daily average vital | Alert if daily average vital |
| | measure is less than _{——}_{——}_{——}% out | measure is less than _{——}_{——}_{——}% out |
| | of acceptable range for more | of acceptable range for more |
| | than _{——}_{——}_{——}# of consecutive days | than _{——}_{——}_{——}# of consecutive days |
| | Measure Out of Range: | Measure Out of Range: |
| | Alert if measure is outside of | Alert if measure is outside of |
| | acceptable range _X_{—} | acceptable range _X_{—} |
| Vital Regimen | Glucose | N/A |
| | Blood Pressure | |
| | Weight | |
| | Pulse Oximetry | |
| | Body Temperature | |
| | Heart Rate | |
| | Respiratory Rate | |
| Units | Fahrenheit (F.) | N/A |
| | Celsius © | |
| Acceptable Range | 96.4-100.3 | N/A |
| Schedule (Frequency) | As Needed | N/A |
| | Daily | |
| | Weekly | |
| | Custom Days | |
| | Custom Weekly Days | |
| Schedule (Time of Day) | Morning, Noon, Afternoon, | N/A |
| | Evening, Bedtime | |
| | Specific Time of Day | |
| | As Directed | |
| Trending Rule(s) | Compliance: | N/A |
| | Alert if compliance is less | |
| | than _{——}_{——}_{——}% over _{——}_{——}_{——} | |
| | consecutive days | |
| | Range: | |
| | Alert if measure is less than | |
| | _{——}_{——}_{——}% out of acceptable range | |
| | for more than _{——}_{——}_{——}# of | |
| | consecutive recordings | |
| | Average Range: | |
| | Alert if daily average vital | |
| | measure is less than _{——}_{——}_{——}% out | |
| | of acceptable range for more | |
| | than _{——}_{——}_{——}# of consecutive days | |
| | Measure Out of Range: | |
| | Alert if measure is outside of | |
| | acceptable range _X_{—} | |

| | Chronic Obstructive Pulmonary | |
|---|---|---|
| Therapeutic Monitoring | Disease (COPD)/Pleural | Chemo Systemic Therapy |
| Template | Effusion with Catheter | (Thoracic Cancer) |
| Wellness Regimen | Exercise | Exercise |
| | Hydration | Hydration |
| | Nutrition | Nutrition |
| | Sleep | Sleep |
| | Bowel Movements | Bowel Movements |
| | Custom | Custom |
| Units | Minutes (exercise) | Minutes (exercise) |
| | oz, ml (Hydration) | oz, ml (Hydration) |
| | Calories (Nutrition) | Calories (Nutrition) |
| | Hours per day (Sleep) | Hours per day (Sleep) |
| | Times per day (Bowel | Times per day (Bowel |
| | Movements) | Movements) |
| | Custom (times per day) | Custom (times per day) |
| Acceptable Range | Daily Range: | Daily Range: |
| | Alert if measure is less | Alert if measure is less |
| | than _{——}_{——}_{——} | than _{——}_{——}_{——} |
| Schedule (Frequency) | As Needed | As Needed |
| | Daily | Daily |
| | Weekly | Weekly |
| | Custom Days | Custom Days |
| | Custom Weekly Days | Custom Weekly Days |
| Schedule (Time of Day) | Morning, Noon, Afternoon, | Morning, Noon, Afternoon, |
| | Evening, Bedtime | Evening, Bedtime |
| | Specific Time of Day | Specific Time of Day |
| | As Directed | As Directed |
| Trending Rule(s) | Measure Out of Range: | Measure Out of Range: |
| | Alert if measure is outside | Alert if measure is outside |
| | of acceptable range | of acceptable range |

### vi. Procedure Regimen Examples

| | Chronic Obstructive Pulmonary | |
|---|---|---|
| Therapeutic Monitoring | Disease (COPD) / Pleural | Chemo Systemic Therapy |
| Template | Effusion with Catheter | (Thoracic Cancer) |
| Procedure Regimen | Post Operative Wound Care | N/A |
| Procedural Tasks | Wound Inspection | N/A |
| Task - Step(s) | Step 1 - Remove Bandage | N/A |
| | Step 2 - Inspect wounds for | |
| | signs of infection | |
| | Step 3 - Treat wound with | |
| | ointment | |
| | Step 4 - Dress and rebandage | |
| | wound | |
| Schedule (Frequency) | As Needed | N/A |
| | Daily | |
| | Weekly | |
| | Custom Days | |
| | Custom Weekly Days | |
| Schedule (Time of Day) | Morning, Noon, Afternoon, | N/A |
| | Evening, Bedtime | |
| | Specific Time of Day | |
| | As Directed | |
| Trending Rules | Compliance: | N/A |
| | Alert if compliance is less | |
| | than _{——}_{——}_{——}% over _{——}_{——}_{——} | |
| | consecutive days | |

### File/Attachment Regimen Examples

| | Chronic Obstructive Pulmonary | |
|---|---|---|
| Therapeutic Monitoring | Disease (COPD)/Pleural | Chemo Systemic Therapy |
| Template | Effusion with Catheter | (Thoracic Cancer) |
| Files/Attachments | Understanding COPD | Understanding |
| | Post Operative Wound care | Chemotherapy |

### vii. Goals Regimen Examples

| | Chronic Obstructive Pulmonary | |
|---|---|---|
| Therapeutic Monitoring | Disease (COPD)/Pleural | Chemo Systemic Therapy |
| Template | Effusion with Catheter | (Thoracic Cancer) |
| Goals | Goals to be achieved: | Goals to be achieved: |
| | Required treatments and | Required treatments and |
| | services including patient | services including patient |
| | actions: | actions: |
| | Arrangements for treatments/ | Arrangements for treatments/ |
| | services (when, who, contact | services (when, who, contact |
| | details): | details): |
| General Instructions | Goals to be achieved: | Goals to be achieved: |
| | Check catheter site daily | Track symptoms weekly |
| | Required treatments and | Required treatments and |
| | services including patient | services including patient |
| | actions: Inspect for catheter | actions: Take weekly |
| | site infection. Track for | symptom survey and track for |
| | symptoms and side effects to | side effects to chemotherapy. |
| | therapy. | Arrangements for treatments/ |
| | Arrangements for treatments/ | services (when, who, contact |
| | services (when, who, contact | details): Call doctor's office |
| | details): Call doctor's office | if any issues arise from |
| | if any issues arise from | treatment |
| | treatment | |
| Lifestyle | Goals to be achieved: Smoking | Goals to be achieved: N/A |
| | Cessation | Required treatments and |
| | Required treatments and | services including patient |
| | services including patient | actions: N/A |
| | actions: Group Therapy and/or | Arrangements for treatments/ |
| | Hypnosis | services (when, who, contact |
| | Arrangements for treatments/ | details): N/A |
| | services (when, who, contact | |
| | details): Sponsor | |
| Biomedical | Goals to be achieved: Track | Goals to be achieved: Track |
| | your biotelemetry (sensor) | your biotelemetry (sensor) |
| | data closely. | data closely. |
| | Required treatments and | Required treatments and |
| | services including patient | services including patient |
| | actions: Call your medical | actions: Call your medical |
| | team if vital measures are | team if vital measures are |
| | outside of threshold. | outside of threshold. |
| | Arrangements for treatments/ | Arrangements for treatments/ |
| | services (when, who, contact | services (when, who, contact |
| | details): Call MGH | details): Call KCI Oncology |
| | Pulmonology team. | team. |
| Complications | Goals to be achieved: Reduce | Goals to be achieved: Reduce |
| | side effects to therapy. | side effects to therapy. |
| | Required treatments and | Required treatments and |
| | services including patient | services including patient |
| | actions: Track side effect and | actions: Track side effect and |
| | disease complications. | disease complications. |
| | Arrangements for treatments/ | Arrangements for treatments/ |
| | services (when, who, contact | services (when, who, contact |
| | details): Call your medical | details): Call your medical |
| | team or ambulatory patient | team or ambulatory patient |
| | center for assistance. | center for assistance. |

### C. Functions

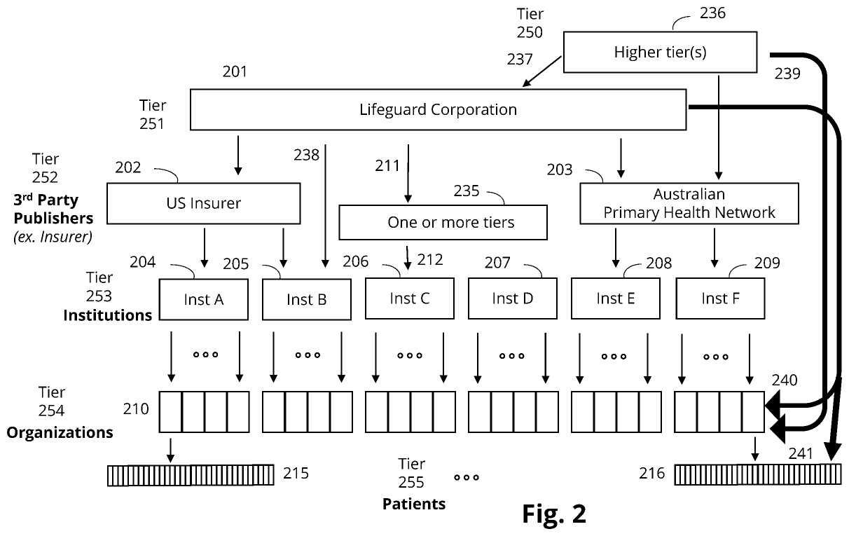

Discussion now turns to the functions available to participants with respect to policy templates. Referring to FIG. 2, in most embodiments, the system provides select or authorized participants the ability to perform at least five core functions with respect to a policy template: create, publish, customize, prescribe and/or use policy templates.

### i. Author/Create

A participant may author or create a policy template. In some embodiments, the system provides tools to enable authoring or creation. An example process for creating a policy template is discussed in detail below. In one or more embodiments, the tools for creating policy templates include an extensible form or API to facilitate creation of a policy template by assembling basic policy template elements, such as regimens for symptoms, vitals, medication, wellness, procedures, files/attachments, and goals. In some embodiments, policy template elements may also include without limitation: description of health service protocols such as therapies and medications; quality of health service measurements, such as patient statuses for surveillance; alert and notification criteria including the identity or nature of entities or persons to receive communications as well as thresholds for sending those communications to each participant or other recipient; authorizations and restrictions for the template to be viewed, requested, subscribed, movement tracked, customization/edit tracked, or associated data tracked.

### ii. Publishing

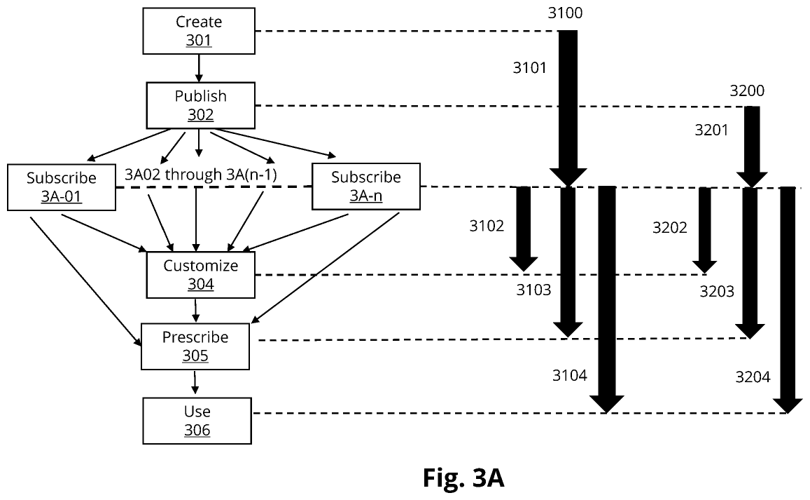

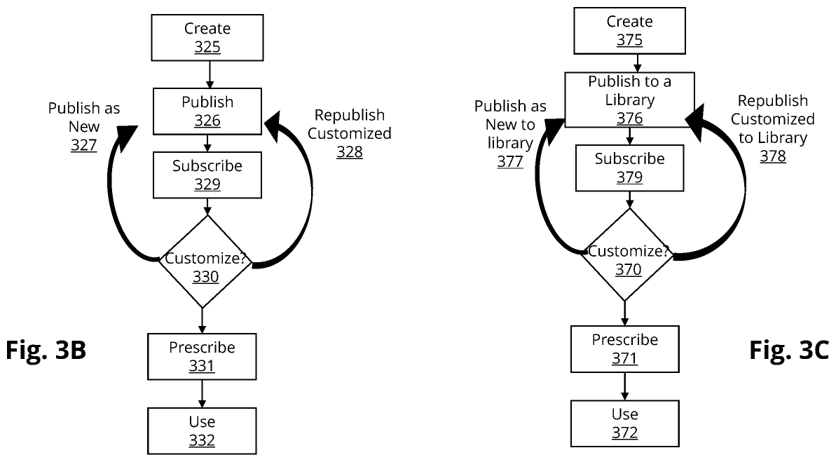

According to certain embodiments, any participant with access to a policy template may publish the template, thereby making it available for other participants to subscribe. In some embodiments, a collection or aggregate of published policy templates make up a library available for other participants to find desired policy templates and subscribe to them. A library may be proprietary or part of an open-source community, where for example creation may be facilitated but sharing may be a requirement. With reference to FIG. 3C, there is shown flow diagram illustrating one or more paths traversed by a policy template in operation of certain embodiments. In particular, at 375, a template is created. Once created, at 376 the template may be published to a library. Once in an accessible library 376, a participant may subscribe 379 to the policy template. Having subscribed 379, a participant may customize 370 a template. In some embodiments, after customizing 370 a policy template, a participant may decide whether to re-publish the customized policy template back to library 376 or another library. Differing embodiments contemplate at least two ways to publish a customized/edited template to a library 376. In one type of embodiment, the customized template is re-published 378 with its origins intact, meaning the system tracks that policy template through the republication and any subsequent subscriptions, tracing it back to when it was a newly created policy template. Another type of embodiment allows the customized policy template to the published to a library 377 as if it was a new template. In this case, the policy template will not be tracked in the same way as path 378, at least in that policy template may be given a new version number and categorized as new and/or tracked as new in the view of ordinary participants. In some embodiments, the system may still track the policy template back to its first creation origin, but the tracking information regarding its new creation will be maintained as well and available for use with the relevant participants. After customization 370, or even if customization 370 is skipped or bypassed as contemplated by certain embodiments, a participant may prescribe the policy template 371. After prescription 371 of the policy template, it may be used 372.

In some embodiments, publishing participants may require attribution for the publication and/or may confine the universe of subscribers, for example, either through whitelisting (i.e., allowed-listing) or blacklisting (i.e., excluded-listing). In other embodiments, the system may confine the ability to publish to certain specific participants or groups of participants, or participant types, such as institutions, insurers, research entities, etc.

Subscribe. Many embodiments allow any participant to subscribe to available published policy templates. Once subscribed, the subscribing participant may prescribe, customize, and/or republish the template. In some embodiments, when a particular participant subscribes to template that was previously published by a second particular participant, if the second participant customizes or edits the template, the changes may automatically flow to the first participant's copy. For example, if a doctor's office subscribes to a particular template and the publisher of that template later makes changes to the template, those changes will automatically appear in the template copy held by the subscribing doctor's office. In addition, a policy template may be sequentially subscribed and customized/edited, and any changes/updates may flow down any number of levels. For example, for first particular template, published by a first participant, any number of participants may subscribe to that first template and then automatically receive updates when the first participant customizes/edits the first template. As another example, the first particular template might be subscribed and customized by a second participant; then republished under a different version ID; and subscribed again by a third participant; and, if the first participant customizes/edits the first template, the update may flow down to both the second and third subscribing participants, and infinitely further down if applicable, all while paying attention to and resolving potential data conflicts.

Some embodiments allow a subscriber to prevent the propagation of changes from prior publishers by re-defining the policy template as a newly authored or newly created template, or by designating either the template or specific fields within a template as “protected.”

### iii. Customize

A participant that subscribes to a policy template may customize/edit the policy template in any manner authorized by the system. Embodiments provide for the customizations to automatically update in the policy templates held by all the downstream subscribers of the customized template. Some embodiments only propagate the updates to downstream subscribers if the customized template is re-published. Other embodiments do not provide for propagating customizations to downstream templates that are re-published as new, or are protected, or with respect to fields within a template that are protected.

### iv. Assign or Prescribe to Patients

In most embodiments, healthcare providers prescribe a policy template to patients to facilitate therapeutic care, surveillance, intervention, and reward. In some implementations, embodiments may represent this prescription as a special type of subscription by a patient. Furthermore, many embodiments implement material distinctions between patients or their representatives and other participants in the system. For most embodiments, one material distinction is that patients may not customize/edit a policy template and may not publish it. Another material distinction is the definition of relationship and roles between a patient and any designated representative of the patient. In these respects, varying embodiments may or may not provide access to certain patient data and communication abilities to the patient or the representative or both based upon the designation of the care provider or the policy template.

### v. Policy Template Tracking and Metadata

When participants perform any of the five foregoing functions (create, publish, customize, prescribe, use) policy templates may either change, move between participants, and/or accrue associated data. Various embodiments track all or at least part of that activity. This is an example of metadata that may be embedded (e.g., travel with) with the template itself or simply be associated with the template in a client-server (e.g., database) arrangement or by reference (e.g., a Universal Resource Locator (URL)) to an external source that allows the metadata to be accessed by system and/or participants interacting with a policy template. Metadata might include any data related to the policy templates, although whether or not particular data is maintained as meta data, embodiments of the disclosure contemplate tracking some or all of any data related to policy template activity. For example, tracking data with respect to policy template versions may include, without limitation, all of the following: a unique or other ID for each template; a version ID for each template or copy of a template, a version being defined in some embodiments as template differing from other versions and the originally created or new policy template; time and date of creation; identity of participant that performed creation; time and date of publication; identity of participant that performed publication; time and date of customization; identity of participant that performed customization; time and date of republication, identity of participant that performed republication; time and date of republication as new; the identity of participant that republished as new; time and date of prescription; the identity of participant making prescription; time and date of patient consent and adoption (use); and, identity of patient adopting template. For clarity, embodiments of the disclosure may include many data as part of an identity such as a participant or subscriber identity, for example: personal identity; organizational identity; grouping within an organization (e.g., department); hardware identity; IP address or similar designation; geo-location; network identities; etc.

In addition to the possibility of maintaining any or all of the foregoing data, many embodiments of the disclosure maintain a collection of data containing all policy template versions in the system, and for each version, the identity of each subscriber, prescriber, and patient/user for each version. Finally, in addition, patient and health care provider data may be maintained in the system and correlated with specific versions of policy templates. In some embodiments, the tracking information, customizations/edits, and template-associated-patient-data may be retained by one or more databases or data structures.

### D. N-Tier Structure

Referring to FIG. 2, there is shown an N-tier structure representing a flexible system for creating, publishing, subscribing, customizing, prescribing, and using (e.g., by a patient) policy templates in the context of therapeutic monitoring of healthcare patients. Various and potential participants in the system are illustratively shown as Lifeguard Corporation 201, US Insurer 202, Australian Primary Health Network (“PHN”) 203, Institution A 204, Institution B 205, Institution C 206, Institution D 207, Institution E 208, Institution F 209, organizations 210, patients 215, patients 216, higher tiers 236, and one or more tiers 235. The arrows pointing downward from each tier represent the general downward flow and publication/subscription relationships of policy templates, from higher tiers down to patients.

Referring again to FIG. 2, in one embodiment, Lifeguard Health Networks 201 is a brand and corporate name used by the assignee of this disclosure, but in context of certain embodiments of FIG. 2. LHN 201 may represent a participant or a participant tier in the illustrated structure of an N-tier system for creating, publishing, subscribing, customizing, prescribing, and using policy templates. In some embodiments LHN 201 is a policy template creator and/or publisher, and in at least one embodiment, LHN 201 is also the facilitator of the system providing the N-tier structure and associated software service, which may involve operating one or more networks, and computer servers, potentially including one or more databases or data structures to track and retain policy template information.

### E. Policy Template Information Flow Between Participants

Referring now to FIG. 3A, there is shown a process diagram illustrating an embodiment of how policy template information may flow through the system. At 301, any appropriately authorized participant may create a therapeutic policy template as described above and publish 302 the template also as described above. Depending upon the embodiment, publication may place the template in a library with other templates and possibly other resources. Some embodiments according to FIG. 3A allow any number of authorized participants, e.g., 3A01 through 3An, to subscribe to the published policy template. After subscribing to a particular policy template, a participant may either: (i) customize 304 the template then prescribe 305 it; or (ii) directly prescribe 305 the template. After the template is prescribed 305, it will be used 306 with the patient as part of therapeutic regimen, patient monitoring, intervention, and/or reward etc.

Referring to FIG. 3B, there is shown another process diagram illustrating a differing embodiment of how policy template information may flow through the system. At 325, any appropriately authorized participant may create a policy template as described above and publish 326 the template also as described above. While not shown in the FIG. 3B, like FIG. 3A, any number of subscribers may subscribe 329 to a published template and then either customize 330 and prescribe 331 that template or directly prescribe 331 it. Referring to FIG. 3B, after a participant customizes 330 a template, there are options 327 to publish the customized template as a newly created template, or simply publish the customized template for others to subscribe. After the template is prescribed 331, it will be used 332 with a patient as part of a therapeutic regimen, patient monitoring, intervention, and/or reward, etc.

Many embodiments of the disclosure include an update feature, so that when a creator or publisher edits or updates a policy template, the updates flow down to all subscribers and/or other users of the template. To implement this feature, some embodiments use one or more databases or data structures to track templates. For example, for any given published template, the subscriber list and associated versions of the template may be tracked.

Referring to FIG. 3A, by way of example, amendment path collection 3100 illustrates the update path for information in one or more embodiments of the present disclosure. In some embodiments, each created policy template is assigned either a locally unique ID or a universally unique ID. If an amendment is made to a template, at the creation level 301, then as shown by portion 3101, some embodiments will propagate that amendment to designated or all subscribed (3A01 through 3An) templates. For certain embodiments, in addition or as an alternative to propagating amendments to the subscribed (3A01 through 3An) templates, the amendments will be propagated via path 3102 to designated or all of the customized 304 policy templates. Furthermore, as an alternative or in addition to propagating create 301 level amendments to one or both of the subscribed (3A01 through 3An) templates and the customized 304 templates, the amendments will be propagated via path 3103, to designated or all of the prescribed 305 policy templates. Finally, for some embodiments, as an alternative or in addition to propagating create 301 level amendments to one, two or all three of the subscribed (3A01 through 3An) templates, the customized 304 templates, and the prescribed 305 templates, the amendments will be propagated via path 3104, to designated or all of the used 306 policy templates.

Referring again to FIG. 3A, by way of another example, amendment path collection 3200 illustrates the update path for information in one or more embodiments of the present disclosure. In some embodiments, each published policy template is assigned either a locally unique ID or a universally unique ID. If an amendment is made to a template, at the publish level 302 (including without limitation embodiments where publication includes republication or publication-as-new of customized policy templates), as shown by portion 3201, some embodiments will propagate that amendment to designated or all subscribed templates (3A01 through 3An). For certain embodiments, in addition or as an alternative to propagating amendments to the subscribed (3A01 through 3An) templates, the amendments will be propagated via path 3202 to designated or all of the customized 304 policy templates. Furthermore, as an alternative or in addition to propagating publish 302 level amendments to one or both of the subscribed (3A01 through 3An) templates and the customized 304 templates, the amendments will be propagated via path 3203, to designated or all of the prescribed 305 policy templates. Finally, for some embodiments, as an alternative or in addition to propagating create 301 level amendments to one, two or all three of the subscribed (3A01 through 3An) templates, the customized 304 templates, and the prescribed 305 templates, the amendments will be propagated via path 3204, to designated or all of the used policy templates 306.

For clarity, the amendment discussion with respect to FIG. 3A is intended to apply equally to the embodiments of FIG. 3B and FIG. 3C and all embodiments where amendment propagation may be useful.

### F. Propagation of Amendments

Depending upon the embodiment and potentially the physical configuration of the system, propagation of amendments may take any practical form. In some embodiments, each template to be updated is replaced with a new version of the template incorporating the amendments. In another embodiment, each template to be updated is amended in accordance with the amendments to be propagated. In yet another embodiment, where differing versions of a template are represented by a base template and differential data, the differential data may be edited to reflect the amendments to be propagated. In one particular embodiment, all (or a group of) the participants in the system share access to a common resource (e.g., database, distributed ledger, blockchain, etc.) and the updates may be propagated by designating a new version of the amended template and further designating the association between that new version and each participant for which an updated version is required. In yet another particular embodiment, all (or a group of) the participants in the system share access to a common database resource and the updates may be propagated by designating a new version of the amended template in association with differential data defining that version; and further designating the association between that new version and each participant for which an updated version is required.

### i. Filtering Amendments

Some embodiments of the present disclosure contemplate selective propagation of amendments. For example, in some embodiments, the participant performing the amendment may designate by precise participant, type of participant (e.g., insurer, institution, research entity, healthcare provider, patient), or relative tier of participant (e.g., flowing up or down) where amendments may propagate. In a more refined embodiments, each amendment or type of amendment may be designated for propagation in the same way. Furthermore, the system facilitator may similarly restrict propagation or, in addition, do so by design of the system (e.g., by design only provide for selected amendment fields or types to propagate to selected participants (e.g., selected by level of authority/access), or types of participants.

### ii. Conflict Resolution

The ability of participants to serially apply amendments or customizations to the same base policy template creates a possibility for conflicts. For example, two customizers may inconsistently edit the same template portion. As another example, a creator or publisher might apply an amendment to a certain template that a downstream prescriber has customized in an inconsistent manner. In either example, or any other example where conflicts may occur, embodiments provide for conflict avoidance and resolution.

In one embodiment, conflicts are avoided by design. For example, in a system where customized policy templates cannot be republished or can only be republished as new templates, conflicts may be avoided by designing the amendment capabilities to be mutually exclusive with the customization capabilities (e.g., a template holder might only be able to amend portions of a policy template that are not subject to propagation of a publisher or creator's customization). Another example of avoiding conflicts is to determine the propagation of amendments prior to the entry of those amendments, and either: restrict the amendments to those that do not interfere with the destination template's data; do not propagate amendments to destination templates where there will be a conflict; or provide warnings to the amending participant regarding conflicts as the amendments are entered. In addition to avoiding conflicts, several embodiments contemplate resolving conflicts. For example, participants having a template that conflicts with an amendment may be notified and given the ability to review/accept/reject each part of any amendment, prior to the amendment being applied.

### iii. Ability to Protect a Template or Reject Amendments

Some embodiments of the present disclosure allow any or all of subscribers, customizers, prescribers, or users to designate a template not to be amended, or not to be amended without permission. In practice, embodiments of the present disclosure may implement this feature by noting the participants intent in one or more database fields that are checked prior to applying amendments. One example of this feature might be where a healthcare provider tailors (i.e., customizes) a template for the needs of a particular patient or patient cohort. Embodiments of the disclosure allow that healthcare provider to protect those customizations using one or more techniques, such as: using one or more menu selections or commands to designate the customizations as protected and ineligible to be changed or replaced other than with the authority of the customizer; using one or more menu selections or commands to require a password or approval code to amend or replace a customized portion; using one or more commands or menu selections to define conditions for rejection or acceptance of customizations.

### G. Tiering According to Organization Type

While a tier in the N-tier system may be represented by any type of participant with a relevant role (e.g., creator, publisher, customizer, subscriber, or patient/patient representative), as shown in FIG. 2, some embodiments contemplate an aspect of tiering as characterized by a participant's role in the healthcare system or the inventive system. For example, FIG. 2 shows LHN 201, which may in certain embodiments, be the system facilitator, a template creator, and a publisher. Above LHN 201 is higher tiers 236, which represents the potential presence of any practical number of tiers above LHN 201. In one embodiment, a participant 236, may be an expert institution that creates and or publishes templates based upon its expertise or research. For example, participant 236 could be Underwriter Laboratories or the U.S. National Institute of Health. In one or more embodiments, Tiers below LHN 201 may include third party publishers 252 such as insurers 202, or national health networks 203. The next illustrative tier down 253 is an “institution” tier 253 having illustrative institutions 204, 205, 206, 207, 208, 209. In many embodiments, institutions may be high order entities or governing bodies such as Centers for Medicaid and Medicaid Services, National Institute of Health, National Cancer Institute; generally governing bodies; IDNs (integrated delivery networks, which are combinations of payer and provider) such as Kaiser or HCA (Health Corporation of America).

Below the institution tier 253 there is an organizations tier 254, which is commonly comprised of healthcare providers. As contemplated in some embodiments, organizations 254 sometimes have affiliations with institutions, and more commonly subscribe to templates than publish them. These may be private practices, primary care networks, healthcare services organizations of all types, or independently insured corporations or other entities.

### H. Flexible Tiering

Referring again to FIG. 2, the participants shown appear in 6 visually intuitive participant tiers 250 through 255. Higher tiers 236 is shown at the top in tier 250, and as suggested by its moniker, represents any number of participant tiers above the tier 251 (containing Lifeguard Health Networks (“LHN”) 201). Below tier 251 is participant tier 252 described as “third party publishers,” which includes US Insurer 202 and Australian PHN 203. Below the third-party publisher tier 252 is participant tier 253 described as institutions. It includes institutions A through F labeled respectively as 204 through 209.

Referring again to FIG. 2, item 235 labeled “one or more tiers” represents the concept present in most embodiments that participant tiering is not a rigidly sequential hierarchy. For example, as indicated by item 235 “one or more tiers,” any practical number of tiers may exist between LHN 201 (in participant tier 251) and the participants 204 through 209 (in participant tier 253); this is so even though simultaneously either US Insurer 202 or Australian PHN 203 each only represent a single tier separating the same span between tier 251 and 253. Similarly, flow arrows 238 and 239, 240, and 241 show how tier skipping is envisioned by many embodiments.

Referring again to FIG. 2, one aspect of the “N-tier” nature of the system is evident in that many embodiments of the present disclosure contemplate the conceptual ability to have many, many tiers (e.g., infinite tiers), in respect to the flow of policy templates between participants and/or types of participants. By way of one potential embodiment, one participant (e.g., 236) may create a policy template, and participant 236 or other participants (e.g., 201, 203, etc., possibly through the use of a library) may publish that policy template, with or without some level of customization (see FIGS. 3A, 3B and 3C above). Thus, in some embodiments, a participant may create and/or publish policy templates that may be customized by downstream participants repetitively and/or sequentially, and depending upon the embodiment, with each customization, movement, and participant being tracked by one or more data structures or databases.

Continuing the illustrative example, participants 204 through 210 may subscribe to one of the published policy templates, e.g., published by 201, 203, or 236. In some embodiments, illustrative policy template subscribers 204 through 210 may customize the template or not, and then either republish it further for use by organizations 210 (e.g., a healthcare provider) or prescribe it to one or more patients 215 and/or 216. As illustrated above, depending upon the embodiment, the policy templates may be propagated downstream either as newly created templates or as policy templates created and/or first published by upstream participants. Thus, differing embodiments of the disclosure may include options for: a single publication followed by one or more subscriptions plus customizations and prescriptions; or, multiple publications of the same original template with potentially stacked (sequentially applied) customizations; or, multiple publications of the same original template with potentially stacked (sequentially applied) customizations, however, where some of the publications are performed as if the customized or sequentially customized template were newly created. A potential beneficial distinction between publishing a template as newly created or not, at least in some embodiments, relates to the tracking of a template's movement and customizations, and the appearance of the template as new. Therefore, one utility of publishing as a customized or “new” policy template may depend upon the participants' business and legal arrangements as well as patient relationships. For example, the decision to publish as new or customized may depend upon branding, data security, data privacy, IP ownership, or confidentiality arrangements as well as other factors.

Continuing the illustration, in at least one embodiment, one or more of policy template subscribers, illustratively 204 through 210, may customize a policy template and publish the customized version as if a newly created policy template, and in turn, downstream participants could do the same, customizing formerly published policy templates and publishing the customized version as new originally created policy templates. In nearly any embodiment, new customizations would be possible from participants as the policy template flowed down through any practical number of tiers. Whether or not a particular embodiment allows policy templates to be republished as newly created after customization, the “N-tier” nature of the concept provides the ability to track the policy template through any practical number of tiers (e.g., up to infinity). Regardless of whether customizations and uses of policy templates are tracked, embodiments envision the capability of providing individualized or group level authorization for access to selected data items. For example, in some embodiment certain select data may flow up the tiered hierarchy (e.g., to creators and publishers) and other specific information may flow down from the publishers and creators to the subscribers and users.

### I. Patients

Like illustrative participant example 235, the institution level 204, 205, 206, 207, 208, 209, or any other level, may exist in any practical number of tiers depending upon industry structure or cooperative nature of the participants. In similar fashion, organizations 210 may exist in one or many tiers, and patients 216, 215 may be assigned policy templates and therapeutically monitored from any other tier having appropriate care giving abilities. Some embodiments even provide for tiering at the patient level, so that patients may help each other in a social networking format or create tiers for different classes of patient representatives that care for the patient and can control certain template specifics that flow into the patients' hands. In other words, in some embodiments, policy templates may flow vertically in any of the intuitively illustrated tiers shown in FIG. 2.

### J. Customization

As introduced above, embodiments of the present disclosure provide for participants to customize templates. The inventive concept is not limited in this regard and all manner of customization is contemplated, and in this context, the word “customize” is intended to include any type of editing or alteration. As a practical matter, however, most embodiments involve customization of elements that bear on therapeutic surveillance and treatment as well as all forms of communication (e.g., alerts and notifications) associated therewith. In many embodiments, the extent of customization may be controlled by one of more of: the system (i.e., software); the policy template creator or publisher; or an upstream subscribing participant. One example of customization is an insurance company or healthcare provider customizing a policy template to alter one or more of: alerts or notification recipients; thresholds for creating alerts or notifications; treatment regimens; data access attributes; etc.

In many embodiments, customizing a policy template may include altering a policy template through selection of available customizations or edits. For example, in one or more embodiments, the creator or publisher of a policy template may define editable portions of the template, including all or no part of any particular template. Depending upon the embodiments, such editable portions may allow for free form customization or merely selection from a menu of customizations made available by the creator or publisher. In some embodiments, the creator or publisher can define portions of the policy template as free form customizable and other portions as customizable only by selection. In addition, some embodiments allow a subscriber to redefine customization parameters prior to republishing or further propagating a policy template. For example, a subscriber might customize and completely lock a policy template prior to propagating it downstream to another participant, such as a patient or patient representative. As described above, customizations along with all activity related to policy templates, their propagation, customization, and use, may be tracked using one or more databases or data structures.

### K. More Embodiments Regarding Higher Level Tiers

Referring again to FIG. 2, above LHN 201 is higher tiers 236, which represents the potential presence of any practical number of tiers above LHN 201. For example, multiple tiers might exist in a community of medical research or expert organizations, or potentially an open-source community; in either case, policy templates may be created and propagated through a series or customizations to create both a very diverse set of extremely specialized policy templates, and any number of policy templates that each benefit from the very specific expertise of multiple participants. One potential example is a policy template that regards a particular diagnosis for a specific heart-related illness. A general heart-oriented research institution might create and publish a policy template regarding that heart related illness. Subsequently, that template might be separately and sequentially customized (e.g., improved) by different organizations that have particular expertise in aspects of heart health relating to the policy template diagnosis. Those aspects might include: kidney-heart relationship; nutrition; coronary artery endothelial health; arterial aneurysms; statins; blood pressure; exercise; body mass index goal; etc. Alternatively, rather than refining the same diagnosis template, the original heart-illness-related template might be revised one or more times either sequentially or in parallel to yield one or more policy templates related to more refined diagnoses, with additional and more refined regimens. In other words, movement through the tiers may result in any number of new, potentially more refined and improved diagnoses policy templates.

### L. Physical Data Flow

With reference to FIGS. 2, 3A and 3B, select embodiments of the present disclosure contemplate that communications between any two participants or group of participants may be made and maintained through any known communication technique. Depending upon the embodiment, databases or other data structures may be used and either centrally maintained or distributed among select participants in the system or select third parties. Sample connections or communication links are shown in FIG. 2 arrows illustrated by 241, 240, 239, 238, 237, 212, 211 and other arrow formed graphics on FIG. 2. The connections are intended to represent any type of communication, such as by traditional IP communications protocols or by a system of access and authorities in a database where a group or all of the participants have some level of access.

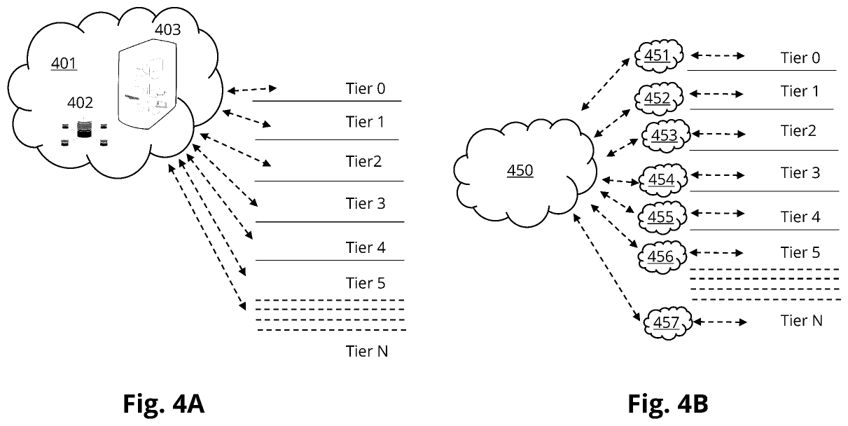

Referring now to FIG. 4A, there is shown an illustration of an embodiment for physical data flow. Cloud 401 represents a server/data resource including one or more server computers 403 and one or more databases 402, including system application software and industry standard server and database operating platforms. The configuration in FIG. 4A represents embodiments in a traditional client server format, where each participant, regardless of tier transacts and communicates with application and/or database software at the server. In this configuration, all activity, e.g., facts about the policy templates, their propagation, amendments, customization, uses, and associated data, may be tracked in a very detailed manner because all activity occurs at the server. Similarly, access to data may be controlled very precisely because a single system administrator and set of application software controls all the activity. For clarity, cloud resource 401 may be a public cloud resource, or dedicated data center resource or a combination of both.

Referring to FIG. 4B, there is shown an alternative embodiment for physical data flow, where each of the cloud resources 450 through 457 is intended to physically be as described with respect to 401 of FIG. 4A. Embodiments consistent with FIG. 4B's configuration allow each participant, tier, or group of participants to have their own set of application software and database. This type of configuration allows participants or groups of participants to shield or withhold data and other information from other system participants. While not shown in the diagram, a participant's or group's dedicated cloud such as 451 may hold patient data, customizations, amendments, or other proprietary data, while synchronizing desired information with the system facilitator's cloud 450. Also, while not depicted, any group of participants, even if in different tiers may share a dedicated cloud, and communications directly between dedicated clouds 451 through 457 is contemplated by varying embodiments. Additionally, a distributed ledger system can be used between any group of participants, even if in different tiers.

### M. Security

In many embodiments data security techniques are used to maintain privacy and/or confidentiality of various stakeholders in the system, including without limitation participants, patients, and patient representatives. Many embodiments employ at least the two general security techniques of access restriction and encryption.

### i. Encryption

Some embodiments require encryption applied to virtually all data in the system, such as policy templates, names or identifying information regarding participants, and patient data such as PHI. Other embodiments allow a system administrator to specify data types for encryption. Still other embodiments allow participants to specify data types for encryption. Yet other embodiments only require encryption of PHI and may provide optionality to the system administrator or participant as discussed above.

Embodiments of the disclosure contemplate the protection of “protected health information” (“PHI”) or other health related information that participants may consider sensitive. For example, embodiments may treat template customization, or the prescription of a template as sensitive information (e.g., PHI). In one embodiment, the system protects (e.g., through encryption and/or sharing restrictions) information for which legal and regulatory authorities require protection. In other embodiments, protection may be more broadly applied than required by law or regulation. For example, since templates are readily relatable to treatments and illness, the sharing of template distribution (or any data that associates individuals with particular templates) may create disclosures that range from extremely sensitive (e.g., implying that a particular patient is being treated for a particular illness) to likely legally not sensitive but perhaps troubling to some patients or healthcare providers (e.g., implying that a certain healthcare provider has one or more patients being treated for a range of potential illnesses). Embodiments of the disclosure provide the ability to protect information in either or both situations. Thus, most embodiments provide protection for data exposing prescription and/or distribution of a template to a patient or patient representative, or for exposing any other situation where the customization, assignment or movement of a template creates an implication regarding a patient or relevant (e.g., identifiable) cohort of patients.

By attending to the security of sensitive information, embodiments of the disclosure allow for distributing templates to individuals and entities that are governed by different customs, regulations and/or laws. By securing data, including templates, during transmission and at rest (where necessary), embodiments may operate across these differing regimens.

### ii. Access

In most embodiments, access to patient data is restricted per the legal and regulatory requirements, and beyond those requirements as requested by authorized participants. For many embodiments, access to patient data is restricted to the provider or care giver team that prescribed the therapeutic monitoring, also referred to as the authoring provider. If the patient has more than one provider, certain embodiments allow for all or designated providers and caregiver teams to be provided access to patient data (e.g., PHI). In addition, embodiments provide for selecting what data will be accessible by a system participant, whether a provider, insurer, institution, patient, patient representative or other. This allows the prescribing provider or another authorized participant to work with the patient to determine the desired distribution of data as to which participants are given access to what types of data or precise data elements.

Many embodiments provide for generation of alerts and notifications based upon therapeutic monitoring relative to thresholds built into policy templates or set by a prescribing or authorized healthcare provider. In one embodiment, the prescribing participant (typically a healthcare provider) may designate access and/or destination for alerts and notifications. In situations where there are multiple participants (e.g., healthcare providers) with access to the monitoring information, some embodiments may allow for the alerts and notifications to go to all such providers, or alternatively allow either the mutually exclusive division of alerts and notifications among providers, or the designation of each alert and/or notification to any number of providers and/or teams.

In most embodiments, creators or publishers do not receive rights to patient data, unless they are also a healthcare provider or care giver. However, in some embodiments, data may be selected by field or a group of fields for access or provision to creators and publishers, typically with patient consent. These instances may occur, for example, in the case of clinical or another research. To facilitate these embodiments along with patient privacy, a mode of some embodiments may provide for automatically anonymizing data sets, and/or filtering data sets to provide the researching creator or publisher only necessary elements.

### iii. Attribution

In certain embodiments, policy templates include attribution for each of one or more of creators, publishers, or customizers in the distribution lineage of a particular policy template. The attribution may be in visible text, meta data and/or retained in a data structure or database that tracks the policy template through a unique identifier. In some embodiments, the system provides an ability to mine insights from data regarding the creation, publishing, customization, subscription, and prescription of policy template. An attribution tag provides a searchable aspect that may increase the available insights.

### iv. Authority/Approvals

Some embodiments allow for the use of authorities and approvals to control the functionality provided with respect to one or a group of policy templates. In one or more embodiments, through either the creation of publish functions, a policy template may specify (e.g., through whitelist/approve-list or blacklist/exclude-list) participants allowed or not allowed to perform one or more of subscribe, customize, re-publish, prescribe, or use the template. Other embodiments provide for an approval process whereby during the creation or publication process, policy templates may be designated as requiring a certain approval before allowing one or more of subscribing, customizing (e.g., editing), re-publishing, prescribing, or using a particular template or group of templates. Depending upon the embodiment, the approver may be specifically designated or default-assigned by either the creator or publisher, for example in their process of creating or publishing. In addition, some embodiments allow for designation of a third party as an approver, such as a particular doctor or a class of participants (e.g., all healthcare providers or all insurers).

Some embodiments allow for the use of authorities and approvals to control the functionality provided to any particular participant. For example, embodiments may provide for designation of functions available on a per-participant basis or by types or other groups of participants. Some embodiments allow participants to be restricted to one or more of creating, publishing, customizing, prescribing, or using policy templates. In certain embodiments, some nuance may be implemented in these restrictions by applying authorities to types of a function. For example, types of publishing can be restricted, such as re-publishing, and re-publishing as new. Many embodiments benefit from the authority capabilities by either preventing unintended or unwise action (e.g., allowing a patient participant to customize or publish), or by creating sales and marketing opportunities by assigning value to each type or level of authority. Depending upon the embodiment, participant authorities may be implemented by designating all authorities applicable to each relevant participant, or by a real-time approval process, whereby approvals are sought from a designated approver proximate to the time of the participant attempting to use a function. For some embodiments, real time approvals are used for one or both of patients and non-prescribing healthcare providers.

In one set of embodiments, a participant (e.g., creator, publisher, customizer, etc.) may choose to provide no authority for certain functions or capabilities. For example, a participant may lock a template so that it cannot be customized or edited by anyone. Similarly, a participant may lock a field or portion of a template so that it may not later be altered by anyone. As one variation of the foregoing, locking a template or portion thereof may not prevent the participant that applied the lock from further editing/changing the template or portion.

Depending upon the embodiment, approvals and authorities may be implemented as follows: an authority provided through the hosting information technology infrastructure, for example Unix or Windows access rights; as a designation in a data record, such as a database or data structure (e.g., a flag for editable or multiple flags indicating who may edit); through cryptography, such as a public/private key system; through physical access restrictions; and any combination of the foregoing or through other known techniques.

### N. More Specific Example Embodiments

As discussed above, embodiments envision that a policy template is constructed as a grouping of regimens. Furthermore, while there are no conceptual limits on the number of regimens some embodiments involve using one or more of the following regimens: symptom assessment; vitals; medication; wellness; procedures; files/attachments; and goals. Also, as discussed above, embodiments incorporate participant and template functionality such as: creating a policy template; publishing a policy template, editing, or customizing a policy template; prescribing a policy template; and, propagating the modifications to subscribed and prescribed policy templates. As discussed above, for many embodiments, logic or design alternatives may be implemented to prevent conflicts between customizations (e.g., from a subscriber) and edits from higher levels (e.g., one or more publishers).

### O. Policy Template Creation

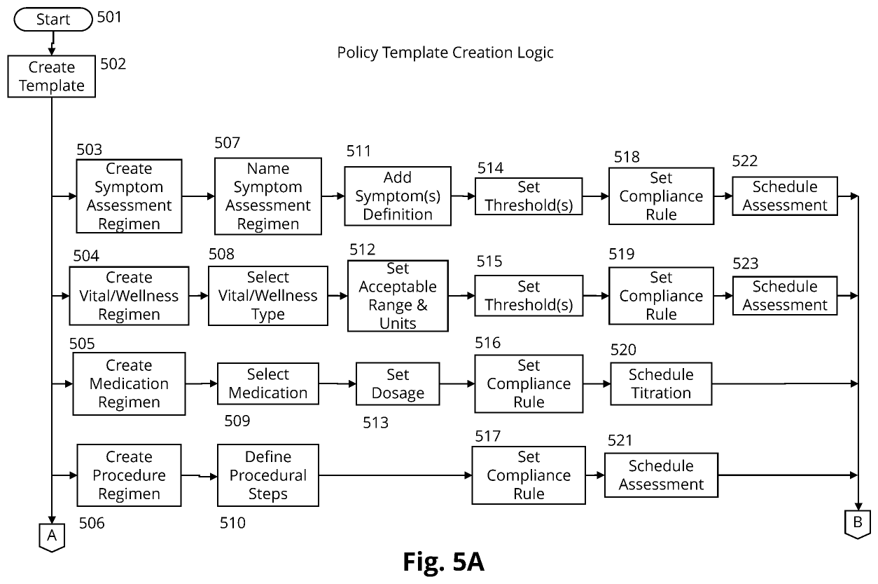

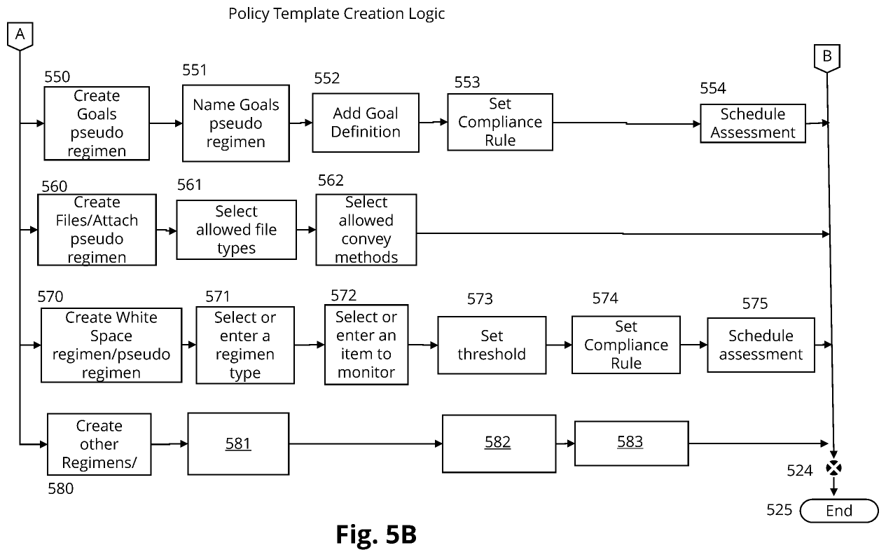

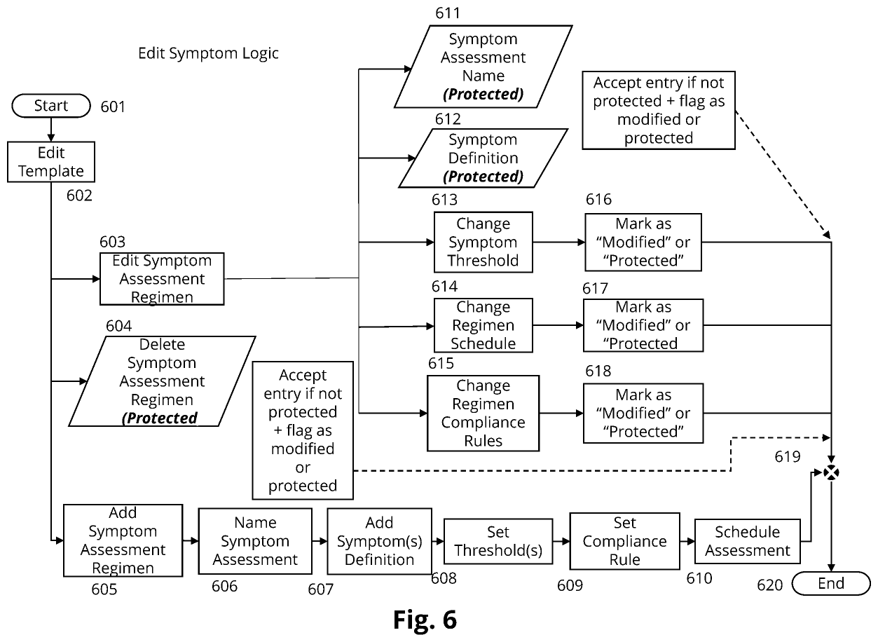

Referring to FIGS. 5A and 5B, there is shown illustrative logic and processes for creating a policy template with at least seven regimens. At 501 and 502 the process starts 501 to create a policy template 502. At 503, a regimen is created, which is a symptom assessment regimen in this example. Some examples of symptom assessments are severity of coughing; pain, dizziness, dry mouth, anxiety, hair loss, or any other human sensation (typically self-assessed) that may be medically evaluated. From 503, attention moves to 507 where the policy template creator names the assessment regimen. This can be a name in multiple languages. For example, if the policy template relates to a diagnosis of COPD, one assessment regimen might be named “COPD Symptoms.” From 507, attention moves to 511 where the policy template creator adds one or more symptom definitions. These can also be definitions in multiple languages. In the example case where coughing is a symptom, the definition may define wet, dry, paroxysmal and croup coughs and provide guidelines for grading severity. From 511, attention moves to 514 to set thresholds. As discussed earlier, thresholds provide a mechanism to trigger the sending of alerts or notifications, which may be sent to any participant or third party, with many embodiments directing them to healthcare providers, patients, and/or patient representatives. Continuing with our example of the symptom coughing, a sample threshold might be severe coughing for three days in a row, or a single day or paroxysmal coughing. Attention next moves to 518 to set compliance rules, which following our coughing example, may illustratively be patient providing the assessment 1 time each day. Finally, at 522, the policy template creator provides an assessment schedule, for example, at 4 PM each day. In some embodiments, the creator allows the patient to choose the time of day they wish to report. Various embodiments allow for the policy template creator to create any number of symptom assessment regimens 503, each presumably correlating with a symptom or a set of related symptoms.

The policy template creation logic and process of FIG. 5A also provides the ability to create one or more vitals or wellness regimens 504. Embodiments of the disclosure may implement a vitals regimen and a wellness regimen as two different sets of regimens. However, FIG. 5A, items 504, 508, 512, 515, 519 and 523 illustrate either regimen set so only one is drawn in the figure, while both are described here. Vitals regimens involve the evaluation of human characteristics that may be sensed (typically, but not exclusively by a device), such as blood pressure, blood oxygen level, EKG, etc. Attention moves to 508 where a vital or wellness type is entered or selected. By way of illustration, the vital type might be blood glucose level. Attention moves to 512 where acceptable range and limits are set, which in our blood glucose example might be between 80 and 250 milligram per deciliter or an analogous range in millimoles per liter. Next, attention moves to 515 to set thresholds, which for our blood glucose example, might be 3 consecutive readings outside of the acceptable range. Next, at 519 a compliance rule is set, which for our blood glucose example, might be acquiring measurements twice each day. Finally, at 523, the schedule assessment specifies when the readings or measurements should be taken, which in the case of blood glucose levels might be upon waking in the morning and after each meal (of course these may be expressed either in explicit time reading or relative to normal human behaviors like meals and rest periods. As with the symptom assessment regimen 503, the create vitals regimen process 504 may be used to create any number of vitals regimens.

Wellness regimens involve the evaluation of health-related lifestyle-oriented activities that patients experience frequently—e.g., daily or multiple times each week. Examples are exercise, hydration, nutrition, sleep, and bowel movements. Absent very close monitoring, for most embodiments, wellness activities are reported by a patient or patient representative. Referring back to FIG. 5A, the flow starting at 504 may be used to describe the creation of a wellness regimen (in addition to the vitals regimens described above). Moving to item 508, selection of a wellness type may be made through a menu, command or by keystroke. By way of illustration, the wellness type might be sleep. Attention moves to 512 where acceptable range and limits are set, which in our sleep example might be between 6 and 10 hours per day. Next, attention moves to 515 to set thresholds, which for our sleep example, might be 3 consecutive readings outside of the acceptable range. Next, at 519 a compliance rule is set, which for our sleep example, might be acquiring the patient's assessment once each day. Finally, at 523, the schedule assessment specifies when the readings or measurements should be taken, which in the case of sleep might be within one hour of waking in the morning (of course this may be expressed either in explicit time reading or relative to normal human behaviors like meals and rest periods). As with the symptom assessment regimen 503, the create wellness regimen process 504 may be used to create any number of wellness regimens.

The policy template creation logic and process of FIG. 5A further provides the ability to create one or more medication regimens 505. Moving attention to 509, a medication may be selected or entered. For the medication regimen creation process, we use the example of Lipitor. Next at 513, a dosage is set, and following our example the dosage may be 20 mg. Moving to a related compliance rule 516, and following our Lipitor example, compliance may be specified at one time per day. Finally, a 520 a titration schedule may be entered. Finishing with our Lipitor example, which titration may be uncommon for a statin, a healthcare provider or other source of medical information may prescribe titration in the form of either loading dose or weaning dose schedules.

As with the other regimens discussed above (e.g., 503 and 504), the create medication regimen process 505 may be used to create any number of medication regimens in the policy template being created.

Moving to 506, the policy template creation logic and process of FIG. 5A provides the ability to create one or more procedure regimens 506. An example of a procedure might be changing a bandage or replacing a catheter or any care procedure that requires one or more steps from beginning to end. At 510, the policy template creator defines the procedural steps. For example, for a bandage change, the steps might be: wet the bandage with alcohol; remove the bandage; clean the affected area with a prescribed cleaning solution; apply a prescribed antibiotic ointment; and apply a new bandage in a specified orientation. Some embodiments provide for embedding instructive multi-media items in the policy template, such as photos and videos. Moving attention to 517, compliance rules may be set. Using the bandage-changing example, a compliance rule might be one bandage change every other day. Finally, at 521 a schedule assessment is entered, which illustratively might be at 10 AM or “after daily bathing.” In some embodiments, the process for creating a procedure regimen may also include a threshold creation. For example, if the patient missed two bandage changes, then the threshold would be exceeded, and an alert issued. Like the regimens discussed above (e.g., 503, 504 and 505), the create procedure regimen process 506 may be used to create any number of procedure regimens in the policy template being created.

Conceptually, embodiments of the present disclosure contemplate the creation of different types of regimens beyond the seven types discussed herein with respect to FIGS. 5A and 5B. In addition, embodiments envision using the creation tool to create regimens or pseudo regimens of many kinds. A pseudo regimen refers to a flow that operates similar to a regimen but does not strictly fit the definition of a health care regimen. With reference to FIG. 5B, item 550 shows a flow for creation of a goal pseudo regimen. At 551 the name of the goal pseudo regimen may be selected or inserted, and at item 552 a goal definition may be selected or entered. A goal may be any milestone or achievement that is desired for the patient. While there are several examples in the chart above, illustratively, goals may include activity (e.g., exercise), compliance (e.g., take your blood pressure or medication as directed), emotional states, medical states, or results, etc. As shown at 553 a goal pseudo regimen may be created with compliance rules (e.g., getting assessed weekly, or engaging in a desired activity daily), which may be entered by selection, keyed entry or otherwise. Moving to item 554, a schedule assessment may be entered, for example to set requirements for when the goal may be assessed.

In a more specific embodiment, from the perspective of a non-patient/representative participant, goal regimens may have many aspects, such as: patient problems/needs/conditions; changes to be achieved; required treatments and services including patient actions; and arrangements for treatments/services (e.g., when, who, contact details, etc.). As shown in the example charts above, each aspect may have related information in the patient-centric areas of: general instructions; lifestyle; biomedical; medications; and complications.

With the aid of these details and the guidance from the figures and the example charts, the skilled artisan will appreciate how the example and other more particular approaches to therapeutic topics may be incorporated into template creation, editing, or reconciliation as described herein.

Referring again to FIG. 5B, there is shown flow 560 for creating a pseudo regimen to a template to the upload or attachment of files. Files may include any kind of media, or written documents that could be useful for the participants such as a patient, patient representative, or a provider. Among the options for creating a files/attach pseudo regimen, are item 561 for selecting or otherwise entering allowed file types (e.g., that may correlate with a participant's ability to interpret files), and item 562 to select or enter allowed methods for conveyance of the file, such as through upload, hyperlink, FTP, and any security desires or requirements.

In addition to the regimens and pseudo regimens discussed above, the process may optionally including the ability to add a white space regimen, where the creator is given prompts to insert the regimen type, an object or event of the interest, any necessary ancillary information about the object/event of interest, any thresholds applicable to the object/event of interest, any compliance rules applicable to the object/event of interest, and any schedule applicable to the object/event of interest.

With reference to FIG. 5B a sample white space flow is shown at 570, where 571 provides for selection or entry of a regiment or pseudo regiment type; 572 provides for selection of entry of an item/activity/patient characteristic to monitor or evaluate; 573 provides for setting thresholds; 574 provides for setting compliance rules; and 575 provides for schedule assessment.

A second white space flow is shown at 580, where the creator's options are as unstructured as possible. In this respect, items 581, 582 and 583 are representative of any number of items that may be entered by the creator with minimal structure imposed by the system. For example, in one embodiment, the system allows for any number of items in the flow and each item allows either manual entry or selection from the full range of selections capable in the system.

Finally with respect to the policy template creation logic and process of FIG. 5A or FIG. 5B, item 524 indicates the inclusion of all completed regimens in the process. For example, every template need not have all seven types of regimens, but the process and logic account for all seven or any quantity ultimately included.

Referring to all the logic and process diagrams associated with this disclosure, the illustrated sequential order of actions/activity is not intended to suggest or imply a restriction in order, but rather only an exemplary illustration. For example, FIG. 5A illustrates 518 “Set Compliance Rule” prior to 522 “Schedule Assessment.” Similarly, 519 “Set Compliance Rule” is shown prior to 523 “Schedule Assessment.” In operation of the system, many embodiments may require the schedule to be set prior to applying a compliance rule, because the compliance rule may enforce the schedule. Of course, FIG. 5A shows the creation of the template; thus, order may not apply if the creating participant has thought through the process in advance, but the principle still applies: the order shown is intended as illustrative and not limiting.

### P. Edit Symptom Logic

Referring to FIG. 6, there is shown a logic and process diagram for editing a policy template. Depending upon the embodiment, the edit symptom logic and process 601 may be applied to templates that are created, published, re-published either as new or otherwise, and/or prescribed. Furthermore, as discussed above, some embodiments call for edits (e.g., customizations) to flow down the tiers, while other embodiments may optionally move edits up, down, or both.

Referring to FIG. 6, items 601 and 602 indicate the start of the edit template process directed toward symptom logic. Item 603 provides for editing a symptom assessment regimen, such as the “COPD Symptoms” example above. At 611, there is a provision to rename the symptom or symptom assessment grouping. As indicated at block 611, many (but not all) embodiments protect pre-existing assessment names and do not allow editing. At item 612, there is a provision for the editing of symptom definition. Like the symptom assessment name 611, the symptom definition 612 is protected and not editable in many (but not all) embodiments. At item 613, a process provides the ability to change a symptom threshold 613. Following the “cough” example, the threshold might be changed from 3 days of severe coughing to 4 days of severe coughing. Alternatively, the threshold might not be changed at all. Moving attention to 616, if there is a change, the data or field is flagged or marked as modified. Also, at item 616, it may be observed that the field or data may additionally or alternatively be marked “protected,” thereby protecting the field from being updated by any future updates or customizations other than those performed by the same editor or creator. This feature allows participants such as healthcare providers in conjunction with their patients to be very careful about changes to protected therapeutic measures.

Moving to item 614, there is a provision for changing the regimen schedule 614 for the symptom. Following the “COPD Symptoms” example, the schedule 614 may be changed to 8 AM and 4 PM or upon waking and before dinner. If there is a change, at 617 the data or field will be marked as modified. Also, at item 617 data may additionally or alternatively be marked “protected,” thereby preventing editing or updates by any future updating or customizations other than those performed by the same editor. This feature allows participants such as healthcare providers in conjunction with their patients to be very careful about changes to protected therapeutic measures. Finally, at 615 the process provides for changing the regimen compliance rules, which in concert with the schedule change may be altered to two-times per day. If there is a change to the compliance rules, then the data or field will be marked as modified at 618. Also, at item 618 data may additionally or alternatively be marked “protected,” thereby preventing editing or updates by any future updating or customizations other than those performed by the same editor. As stated above, this feature allows healthcare providers in conjunction with their patients to be very careful about changes to protected therapeutic measures.

Moving attention now to 604, there is a provision in the process to delete a symptom or symptom assessment grouping in its entirety. In some embodiments, when a symptom is deleted, all historical data is retained at the patient level. However, as indicated at item 604, many (but not all) embodiments protect pre-existing symptoms and do not allow for their complete deletion.

Attention is now drawn to item 605 which shows the add symptom assessment process within the overall symptom edit logic and process. At 606 a new symptom assessment name is entered or selected and received by the system. Next at 607, a definition for the symptom is entered or selected and received by the system. At 608 the editing participant sets thresholds relative to the added symptom, and at 609 compliance rules are set. Finally, at 610, a schedule assessment is entered or selected and received. Notably, some embodiments prevent the addition of new system assessments in order to avoid the new assessment from serving as a loophole to a pre-existing assessments. Furthermore, other embodiments do not allow any specific system assessment additions (and/or in some embodiments amendments) that create loopholes in symptom assessment.

With respect to the edit symptom logic and process of FIG. 6, item 619 indicates the inclusion on all completed activities in the process. For example, every edit symptom operation need not exercise all the ability of the edit template 602 process, but the process and logic account for all that is shown and described. In addition, embodiments allow for editing all the desired symptom regimens in any policy template.

### Q. Edit Vitals or Wellness Logic

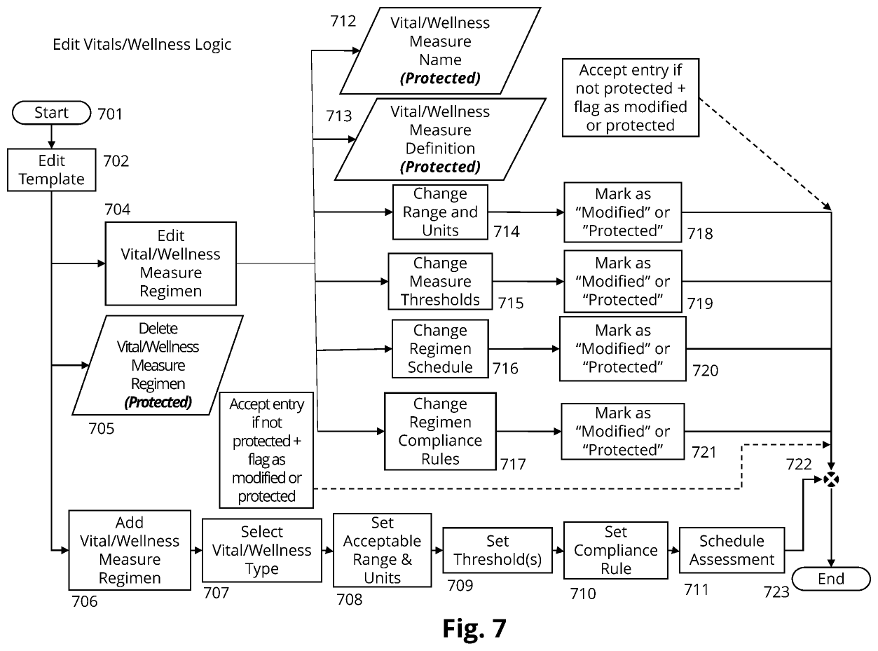

As explained above, vitals and wellness are two different regimen types. However, the same diagrams are used for them because they share common process aspects. Thus, referring to FIG. 7, the diagram will be explained twice-once with reference to vitals and once with reference to wellness.

Referring to FIG. 7, there is shown a logic and process diagram for editing a vitals regimen. Depending upon the embodiment, the edit vitals regimen 701 may be applied to templates that are created, published, re-published either as new or otherwise, and/or prescribed. Furthermore, as discussed above, some embodiments call for edits (e.g., customizations) to flow down the tiers, while other embodiments may optionally move edits up, down, or both.

Referring to FIG. 7, items 701 and 702 indicate the start of the edit vitals regimen process. Item 704 provides for editing a vitals regimen, such as the “blood glucose” example above. Moving attention to item 712, there is shown a provision for editing the name of a vitals regimen. As evident from the item 712 text, many (but not all) embodiments protect the name of the vitals regimen and do not allow edits. Similarly, moving to item 713, the process has a provision for editing the vitals definition 713, although many (but not all) embodiments protect the definition and thus, do not allow editing. Moving to item 714, the item provides for changing range and units. Following the blood glucose example above, the range may be changed to 65 through 200 mg/dl and the units may change if applicable. Moving to 718, if there is a change to the range data, then that data or field will be marked as modified to potentially prevent unauthorized customizations or edits from changing the field. Also, at item 718 data may additionally or alternatively be marked “protected,” thereby preventing editing or updates by any future updating or customizations other than those performed by the same editor or creator. This feature allows healthcare providers in conjunction with their patients to be very careful about changes to protected therapeutic measures.

Moving attention to 715, there is process and logic capability to change measure thresholds. Following the blood glucose example above, the threshold might be changed from 3 consecutive days to 4 consecutive days. Moving to 719, if there is a change to the threshold data, then that data or field will be marked as modified 719. Also, at item 719, data may additionally or alternatively be marked “protected,” thereby preventing editing or updates by any future updating or customizations other than those performed by the same editor. This provides a feature for healthcare providers in conjunction with their patients to be very careful about changes to protected therapeutic measures. Moving attention now to 716, there is process and logic capability to change regimen schedule. Following the blood glucose example above, the regimen schedule might be changed to 10 AM, noon, and 6 PM. Moving to 720, if there is a change to the regimen schedule data, then that data or field will be marked as modified 720. Also, at item 720, data may additionally or alternatively be marked “protected,” thereby preventing editing or updates by any future updating or customizations other than those performed by the same editor. This provides a feature for healthcare providers in conjunction with their patients to be very careful about changes to protected therapeutic measures.

Moving attention to 717, there is process and logic capability to change Regimen compliance rules. Following the blood glucose example above, the compliance rule might be changed from twice each day to three times each day. Moving to 720, if there is a change to the compliance rules data, then that data or field will be marked as modified 721. Also, at item 721, data may additionally or alternatively be marked “protected,” thereby preventing editing or updates by any future updating or customizations other than those performed by the same editor. This provides a feature for healthcare providers in conjunction with their patients to be very careful about changes to protected therapeutic measures.

Referring to FIG. 7, the diagram will now be used with reference to wellness. FIG. 7 shows a logic and process diagram for editing a wellness regimen. Depending upon the embodiment, the edit wellness regimen 701 may be applied to templates that are created, published, re-published either as new or otherwise, and/or prescribed. Furthermore, as discussed above, some embodiments call for edits (e.g., customizations) to flow down the tiers, while other embodiments may optionally move edits up, down, or both.

Referring to FIG. 7, items 701 and 702 indicate the start of the edit wellness regimen process. Item 704 provides for editing a wellness regimen, such as the “exercise” example shown in the chart above. Moving attention to item 712, there is shown a provision for editing the name of a wellness regimen. As evident from the item 712 text, many (but not all) embodiments protect the name of the wellness regimen and do not allow edits. Similarly, moving to item 713, the process has a provision for editing the wellness definition 713, although many (but not all) embodiments protect the definition and thus, do not allow editing. Moving to item 714, the item provides for changing range and units. Following the exercise example above, the range may be changed to increase requirements and the units may change if applicable (e.g., minutes to hours, or temporal to non-temporal). Moving to 718, if there is a change to the range data, then that data or field will be marked as modified to prevent unauthorize (e.g., upstream) customizations from propagating to this field. Also, at item 718 data may additionally or alternatively be marked “protected,” thereby preventing editing or updates by any future updating or customizations other than those performed by the same editor or creator. This feature allows healthcare providers in conjunction with their patients to be very careful about changes to protected therapeutic measures.

Moving attention to 715, there is process and logic capability to change measure thresholds. Following the exercise example above, the threshold (e.g., schedule frequency) might be changed from 3 times per week to 4 times per week. Moving to 719, if there is a change to the threshold data, then that data or field will be marked as modified 719. Also, at item 719, data may additionally or alternatively be marked “protected,” thereby preventing editing or updates by any future updating or customizations other than those performed by the same editor or creator. This provides a feature for healthcare providers in conjunction with their patients to be very careful about changes to protected therapeutic measures. Moving attention now to 716, there is process and logic capability to change regimen schedule. Following the exercise example above, the regimen schedule (e.g., frequency time of day) might be changed to 6 PM or after dinner. Moving to 720, if there is a change to the regimen schedule data, then that data or field will be marked as modified 720. Also, at item 720, data may additionally or alternatively be marked “protected,” thereby preventing editing or updates by any future updating or customizations other than those performed by the same editor or creator. This provides a feature for healthcare providers in conjunction with their patients to be very careful about changes to protected therapeutic measures.

Moving attention to 717, there is process and logic capability to change Regimen compliance rules (e.g., trending rules). Following the exercise example above, the compliance rule might be changed to cause alerting more easily. Moving to 720, if there is a change to the compliance rules data, then that data or field will be marked as modified 721. Also, at item 721, data may additionally or alternatively be marked “protected,” thereby preventing editing or updates by any future updating or customizations other than those performed by the same editor or creator. This provides a feature for healthcare providers in conjunction with their patients to be very careful about changes to protected therapeutic measures.

Turning now to Item 705, there is shown a process and logic capability to delete a pre-existing wellness regimen entirely. As indicated in the text at 705, many embodiments protect pre-exiting wellness regimens, such that deletions are not allowed. Moving now to item 706, there is shown logic and a process for adding a wellness regimen. At 707, the process and logic provide for selection or entering of a wellness type. Moving forward, at 708, acceptable range and units may be entered or selected. At 709, thresholds may be set through entry or selection, and at 710 compliance rules may be set. Finally, at 711, a schedule assessment may be entered or selected.

With respect to the edit wellness logic and process of FIG. 7, item 722 indicates the inclusion on all completed activities in the process. For example, every edit wellness logic 702 operation need not exercise all the ability of the edit template 702 process, but the process and logic account for all that is shown and described. In addition, embodiments allow for editing all the desired vitals regimens in any policy template.

### R. Edit Medication Regimen Logic

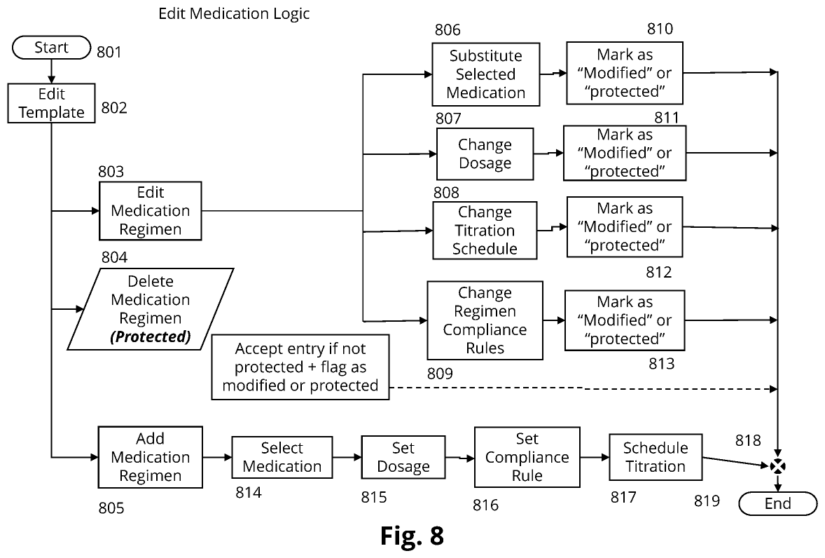

Referring to FIG. 8, there is shown a logic and process diagram for editing a medication regimen. Depending upon the embodiment, the edit medication regimen 801 may be applied to templates that are created, published, re-published either as new or otherwise, and/or prescribed. Furthermore, as discussed above, some embodiments call for edits (e.g., customizations) to flow down the tiers, while other embodiments may optionally move edits up, down, or both.

Referring to FIG. 8, items 801 and 802 indicate the start of the edit medication regimen process. Item 803 provides logic and a process for editing an existing medication regimen. The illustration here follows the example of Lipitor above. With attention to item 806, there is shown a process for substituting a medication. Following the Lipitor example, a substitution might be for a generic Atorvastatin, or for an alternative statin such as Crestor. As shown at item 810, if a modification/substitution occurs then the field or data will be marked as modified. Also, at item 810, it may be observed that the field or data may additionally or alternatively be marked “protected,” thereby preventing the field from being updated by any future updates or customizations other than those performed by the same editor or creator. This provides a feature for healthcare providers in conjunction with their patients to be very careful about changes to protected therapeutic measures.

Moving to item 807, there is shown logic and a process for changing a medication dosage. The illustration here follows the example of Lipitor above, and a dosage change might be from 20 mg to 10 mg. As shown at item 811, if a dosage change occurs then the field or data will be marked as modified. Also, at item 811, the dosage change field or data may additionally or alternatively be marked “protected,” thereby preventing the field from being updated by any future updates or customizations other than those performed by the same editor or creator. This provides a feature for healthcare providers in conjunction with their patients to be very careful about changes to protected therapeutic measures.

Referring now to item 808, there is shown logic and a process for changing medication titration schedule. Following the example of Lipitor above, a healthcare professional may change the loading or weaning schedule. As shown at item 812, if a titration change occurs, then the field or data will be marked as modified. Also, at item 812, the titration change field or data may additionally or alternatively be marked “protected,” thereby preventing the field from being updated by any future updates or customizations other than those performed by the same editor. This provides a feature for healthcare providers in conjunction with their patients to be very careful about changes to protected therapeutic measures.

Turning attention to item 809, there is shown logic and a process for changing regimen compliance rules, and following the Lipitor example, compliance rules may be changed to twice per day. As shown at item 813, if a compliance rule change occurs, then the field or data will be marked as modified. Also, at item 813, the compliance rule field or data may additionally or alternatively be marked “protected,” thereby preventing the field from being updated by any future updates or customizations other than those performed by the same editor or creator. This provides a feature for healthcare providers in conjunction with their patients to be very careful about changes to protected therapeutic measures.

Moving attention now to item 804, there is shown a process and logic capability to delete a pre-existing medication regimen entirely. As indicated in the text at 804, many (but not all) embodiments protect medication regimens, such that deletions are not allowed. Moving attention to 805, there is shown a process and logic for adding a medication regimen. At 814, a medication may be entered or selected. Next, at 816, a dosage may be set through selection of entry, and at 816 compliance rules may be entered or selected. Finally, at 817, titration schedule may be entered or selected.

With respect to the edit medication logic and process of FIG. 8, item 818 indicates the inclusion on all completed activities in the process. For example, every edit medication logic operation need not exercise all the ability of the edit template 802 process, but the process and logic account for all that is shown and described. In addition, embodiments allow for editing all the desired medication regimens in any policy template.

### S. Edit Procedure Logic

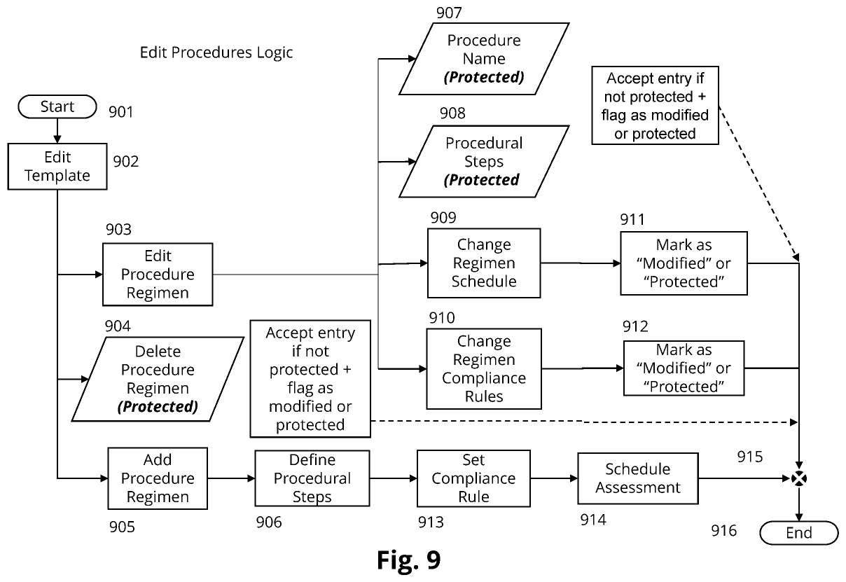

Referring to FIG. 9, there is shown a logic and process diagram for editing a procedures regimen. Depending upon the embodiment, the edit procedures regimen 901 may be applied to templates that are created, published, re-published either as new or otherwise, and/or prescribed. Furthermore, as discussed above, some embodiments call for edits (e.g., customizations) to flow down the tiers, while other embodiments may optionally move edits up, down, or both.

Referring to FIG. 9, items 901 and 902 indicate the start of the edit procedures regimen process. Item 903 provides logic and a process for editing an existing procedure regimen. Moving attention now to item 907, there is shown a process and logic capability to edit a pre-existing procedure name. As indicated in the text at 907, many (but not all) embodiments protect procedure names such that deletions are not allowed. Moving attention now to item 908, there is shown a process and logic capability to edit pre-existing procedural steps. As indicated in the text at 908, many (but not all) embodiments protect procedural steps such that deletions are not allowed.

Moving attention to 909, there is logic and process capability for changing regimen schedule. Following the bandage change example from above, the schedule might change to 8 PM or evening before bed. As shown at item 911, if a modification is made then the field or data will be marked as modified. Also, at item 911, the field or data may additionally or alternatively be marked “protected,” thereby preventing the field from being updated by any future updates or customizations other than those performed by the same editor or creator. This provides a feature for healthcare providers in conjunction with their patients to be very careful about changes to protected therapeutic measures.

With attention to 910, there is shown logic and process capability for changing compliance rules. Following the bandage change example from above, the compliance rule might change to “every day.” Moving to item 912, if a modification is made then the field or data will be marked as modified. Also, at item 912, the field or data may additionally or alternatively be marked “protected,” thereby preventing the field from being updated by any future updates or customizations other than those performed by the same editor or creator. This provides a feature for healthcare providers in conjunction with their patients to be very careful about changes to protected therapeutic measures.

Moving attention now to item 904, there is shown a process and logic capability to delete a pre-existing procedure regimen entirely. As indicated in the text at 904, many embodiments protect procedure regimens, such that deletions are not allowed. Referring now to item 905, there is shown a capability to add a procedure regimen, which follows the logic and process of defining procedure steps 906, setting compliance rules 913, and providing schedule assessment 914, all potentially performed by data selection, keyed entry, or other known technique.

With respect to the edit procedure logic and process of FIG. 9, item 915 indicates the inclusion on all completed activities in the process. For example, every edit procedure operation need not exercise all the ability of the edit template 902 process, but the process and logic account for all that is shown and described. In addition, embodiments allow for editing all the desired procedure regimens in any policy template.

### T. Edit Goals Logic

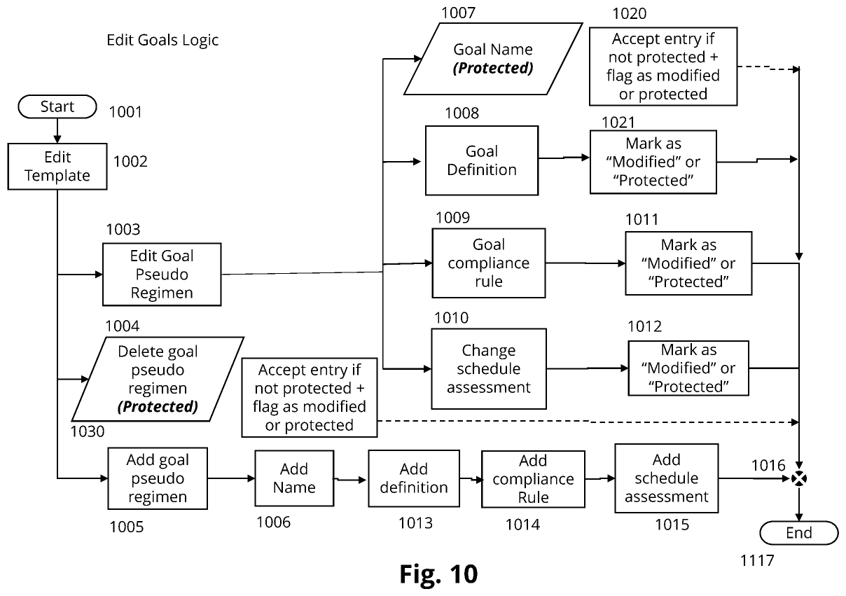

Referring to FIG. 10, there is shown a logic and process diagram for editing a goals pseudo-regimen. Depending upon the embodiment, the edit pseudo-regimen 1001 may be applied to templates that are created, published, re-published either as new or otherwise, and/or prescribed. Furthermore, as discussed above, some embodiments call for edits (e.g., customizations) to flow down the tiers, while other embodiments may optionally move edits up, down, or both.

Referring to FIG. 10, items 1001 and 1002 indicate the start of the edit goals regimen process. Item 1003 provides logic and a process for editing an existing goals pseudo-regimen. Moving attention now to item 1007, there is shown a process and logic capability to edit a pre-existing goals name. As indicated in the text at 1007, many (but not all, e.g., 1020) embodiments protect goals names such that renaming is not allowed. Moving attention now to item 1008, there is shown a process and logic capability to edit pre-existing goals definition, which may be accepted by menu, key entry or by other known mechanisms. As indicated in the text at 1021, if the goal definition is amended then a modified flag or indicator will be activated, and a participant user may activate a protected flag thereby preventing the field from being updated by any future updates or customizations other than those performed by the same editor or creator. This provides a feature for healthcare providers in conjunction with their patients to be very careful about changes to protected therapeutic measures.

Moving attention to 1009, there is logic and process capability for changing a goal compliance rule and/or satisfaction rule, which may be accepted by menu, key entry or by other known mechanisms. As shown at item 1011, if a modification is made then the field or data will be marked as modified. Also, at item 1011, the field or data may additionally or alternatively be marked “protected,” thereby preventing the field from being updated by any future updates or customizations other than those performed by the same editor or creator. This provides a feature for healthcare providers in conjunction with their patients to be very careful about changes to protected therapeutic measures.

With attention to 1010, there is shown logic and process capability for changing a schedule assessment for a goal pseudo-regimen. Moving to item 1012, if a modification is made then the field or data will be marked as modified. Also, at item 1012, the field or data may additionally or alternatively be marked “protected,” thereby preventing the field from being updated by any future updates or customizations other than those performed by the same editor or creator. This provides a feature for healthcare providers in conjunction with their patients to be very careful about changes to protected therapeutic measures.

Moving attention now to item 1004, there is shown a process and logic capability to delete a pre-existing goal pseudo-regimen entirely. As indicated in the text at 1004, many (but not all, e.g., 1030) embodiments protect goals pseudo regimens, such that deletions are not allowed. However, as indicated at 1030, embodiments that allow deletion may follow the same basic procedure as editing shown in this disclosure.

Referring now to item 1005, there is shown a capability to add a goal pseudo-regimen, which follows the logic and process of naming the regimen 1006, adding a definition 1013, adding a compliance rule 1014, and providing a schedule assessment 1015, all potentially performed by data selection, keyed entry, or other known technique.

With respect to the edit goal logic and process of FIG. 10, item 1016 indicates the inclusion on all completed activities in the process. For example, every edit goal logic and process need not exercise all the ability of the edit template 1002 process, but the process and logic account for all that is shown and described. In addition, embodiments allow for editing all the desired goal regimens in any policy template.

As discussed above, all the logic and process diagrams associated with this disclosure provide illustrated sequential order of actions/activity that is not intended to suggest or imply a restriction in order. FIG. 10 provides a similar example to the one discussed with respect to FIG. 5A. Particularly, FIG. 10 illustrates 1009 “Goal compliance rule” potentially prior to 1010 “change schedule assessment.” As discussed above, in operation of the system, many embodiments may require the schedule to be set prior to applying a compliance rule, because the compliance rule may enforce the schedule. Of course, FIG. 10 shows the editing of the template, thus order may not apply if the editing participant has thought through the process in advance, but the principle still applies: the order shown is intended as illustrative and not limiting.

### U. Edit Files/Attachment Logic

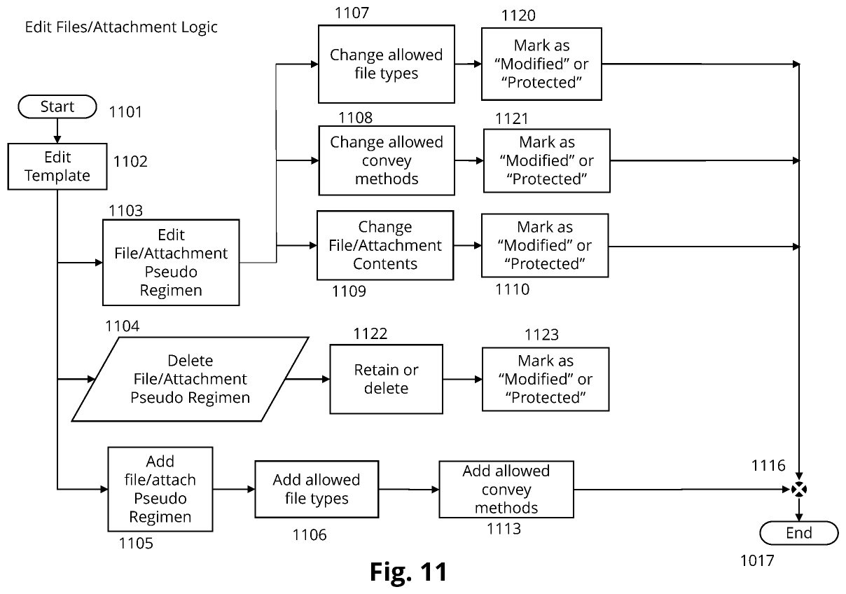

Referring to FIG. 11, there is shown a logic and process diagram for editing a files/attachment pseudo-regimen (e.g., 1103). Depending upon the embodiment, the edit pseudo-regimen 1101/1102 may be applied to templates that are created, published, re-published either as new or otherwise, and/or prescribed. Furthermore, as discussed above, some embodiments call for edits (e.g., customizations) to flow down the tiers, while other embodiments may optionally move edits up, down, or both.

With attention to 1103 there is shown the entry to the process for editing a file/attachment pseudo-regimen. At 1107, there is shown logic and process capability for changing allowed file types for a files/attachment pseudo-regimen. A file type refers to a format or protocol for a file or other content. Example of file types are media file types (e.g., TIFF, JPEG, etc.), business software file types (e.g., XLSX, DOCX, PDF, PPT etc.) or other file types used by consumers or business organizations. In some embodiments, the types of files that are allowable may depend upon the participant performing the editing and the particular capabilities they believe will serve their counterpart participants best (e.g., based upon perceptions of the devices in use or the technical abilities of the counterpart participants).

According to varying embodiments of the disclosure, identities of allowable file types may be stored in any suitable place, including adjacent to any database serving the participants. Furthermore, for embodiments of the disclosure that employ an architecture using a centralized (or apparently centralized) database, file type identities may be incorporated in the database for ready access.

Referring again to item 1107, entry of allowable file types may be received as a menu selection, keyed-entry or by any other known mechanism. Moving to item 1120, if a modification is made then the field or data will be marked as modified. Also, at item 1120, the field or data may additionally or alternatively be marked “protected,” thereby preventing the field from being updated by any future updates or customizations other than those performed by the same editor or creator. This provides a feature for healthcare providers in conjunction with their patients to be very careful about changes to protected therapeutic measures.

With attention to 1108, there is shown logic and process capability for changing allowed convey methods for a files/attachment pseudo-regimen. A convey method refers to a capability to convey file or attachment information from one participant to another. In some embodiments, the types of convey methods that are allowable may depend upon the participant performing the editing and the particular methods they believe will serve their counterpart participants best (e.g., based upon perceptions of the devices in use or the technical abilities of the counterpart participants). Examples of allowable convey methods are as follows: uploading a file; uploading a file through a secure service, such as a secure FTP; emailing a file or preparing a file for emailing on an available email application; providing a reference to a file, such as a hyperlink; providing a reference to a web resource, that may or may not enable access to a file; and, providing an upload capability plus a reference to a file or web resources stored either locally or remotely from the participant accessing the content.

According to varying embodiments of the disclosure, files or other resources made available through the system may be stored in any suitable place, including adjacent to any database serving the participants. Furthermore, for embodiments of the disclosure that employ an architecture using a centralized (or apparently centralized) database, references to the files, attachments or other resources may be incorporated in the database for ready access.

Referring again to item 1108, entry of an allowable convey method may be received as a menu selection, keyed-entry or by any other known mechanism. Moving to item 1121, if a modification is made then the field or data will be marked as modified. Also, at item 1121, the field or data may additionally or alternatively be marked “protected,” thereby preventing the field from being updated by any future updates or customizations other than those performed by the same editor or creator. This provides a feature for healthcare providers in conjunction with their patients to be very careful about changes to protected therapeutic measures.

With attention to 1109, there is shown logic and process capability for changing contents of a file/attachment pseudo-regimen. According to varying embodiments of the disclosure, files or other resources made available through the system may be stored in any suitable place, including adjacent to any database serving the participants. Furthermore, for embodiments of the disclosure that employ an architecture using a centralized (or apparently centralized) database, references to the files, attachments or other resources may be incorporated in the database for ready access.

Referring again to item 1109, changes to the contents of a file/attachment pseudo-regimen may be received as a menu selection, keyed-entry or by any other known mechanism. Moving to item 1110, if a modification is made, then the field or data will be marked as modified. Also, at item 1121, the field or data may additionally or alternatively be marked “protected,” thereby preventing the field from being updated by any future updates or customizations other than those performed by the same editor or creator. This provides a feature for healthcare providers in conjunction with their patients to be very careful about changes to protected therapeutic measures.

Moving attention now to item 1104, there is shown a process and logic capability to delete a pre-existing file/attachment pseudo-regimen entirely, which may be accomplished as a simple selection, or by other know mechanisms. Moving attention to item 1122, a selection may be made regarding whether previously linked or uploaded materials should be retained or deleted. Some embodiments allow selection of retention or deletion separately for each file/resource. The selection may be made through a menu, checkbox or other know mechanisms. Moving to item 1123, if a deletion is selected, then the field or data will be marked as modified. Also, at item 1123, the field or data may additionally or alternatively be marked “protected,” thereby preventing the field from being updated by any future updates or customizations other than those performed by the same editor or creator. This provides a feature for healthcare providers in conjunction with their patients to be very careful about changes to protected therapeutic measures.

Referring now to item 1105, there is shown a capability to add a file/attachment pseudo-regimen, which follows the logic and process of adding allowed file types 1106, and adding allowed convey methods 1113, both potentially performed by data selection, keyed entry, or other known technique.

With respect to the edit file/attachment logic and process of FIG. 11, item 1116 indicates the inclusion of all completed activities in the process. For example, every edit procedure operation need not exercise all the ability of the edit template 1102 process, but the process and logic account for all that is shown and described. In addition, embodiments allow for editing all the desired goal regimens in any policy template.

### V. Other Editing

As shown in FIG. 5B, embodiments of the disclosure illustrate and contemplate white space regimens with minimal structure, so diagrammatic editing processes are not as illustrative as the examples above where structure can be used a mechanism for conveying principles. There are, however, embodiments of the disclosure that contemplate editing functionality for any policy template within the scope of this disclosure and perhaps beyond. The underlying principles for this editing are as follows: Any regimen aspect that is created, may have a process for editing; any edits made should be able to propagate to related policy templates at any tier; meta data may be retained regarding any edits, so for example, the history of edits may be employed; any regimen aspect may have a protection mechanism to prevent updating from changing a regimen (e.g., edited portion) in a manner that was ultimately undesired by a participant, for example, using or prescribing a template having that regimen; any regiment may have the capability of being deleted, even if that capability is modified; an editing system may allow adding a practically unlimited number of regimens; and, the arrangement of allowable and non-allowable edits/modification, or the allowed propagation of those edits may be used to create systems for harmonious synchronization of edits into related policy templates.

### W. Reconciliation Logic

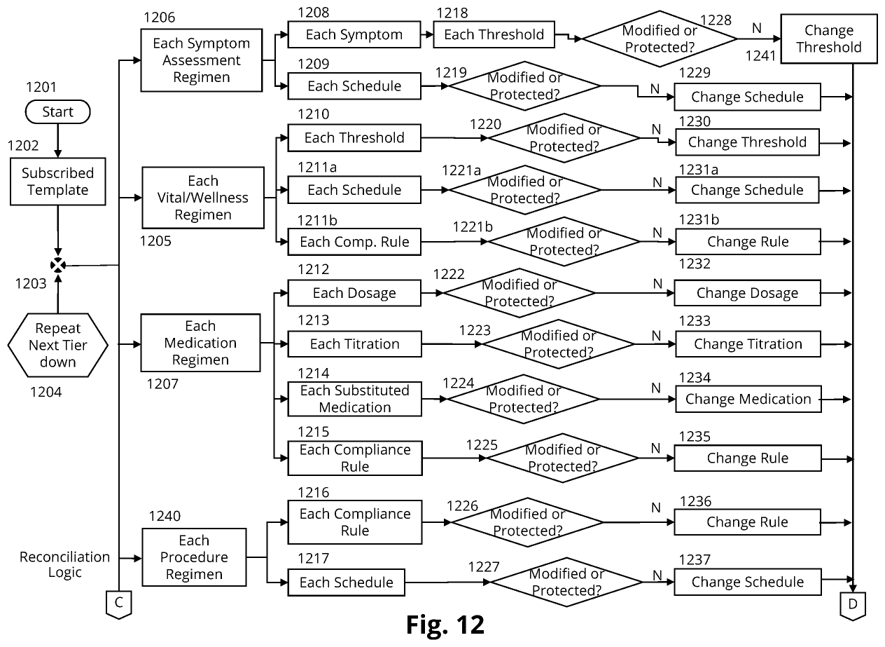

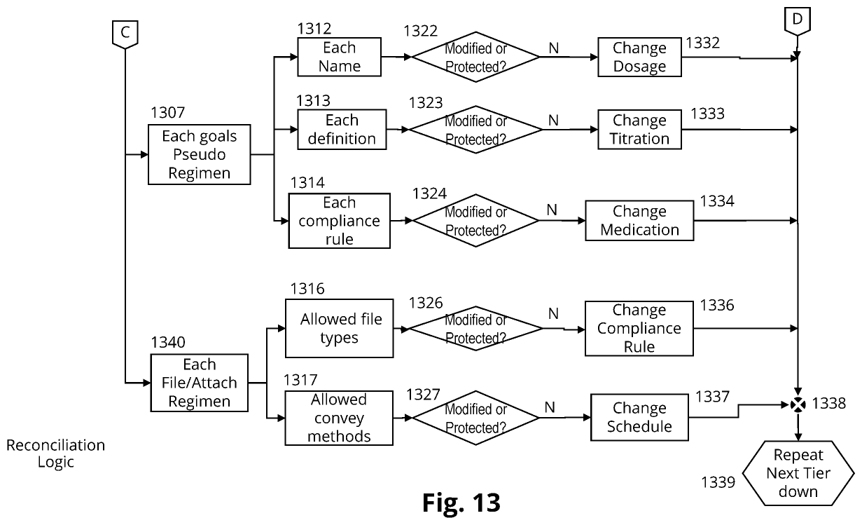

As described above, many embodiments contemplate propagating data updates to policy templates that are subscribed or prescribed and in use (See for example FIG. 3A with particular reference to 3100 and 3200, and associated description). With reference to FIGS. 12 and 13, there is shown a process and logic for reconciling update information with a subscribed or prescribed policy templates. Items 1201 and 1202 indicate the start of a reconciliation process of update information flowing from another participant's (e.g., a publisher's, re-publisher's, etc.) update of the same template (see for example FIG. 6, FIG. 7, FIG. 8, FIG. 9, FIG. 10, and FIG. 11 regarding template editing), generally from a higher-level tier to a lower-level tier. Some embodiments employing concepts from all of FIGS. 6, 7, 8, 9, 10, 11 and 12, provide a system whereby infinite tiers of participants may share policy templates and customizations without conflict.

The reconciliation process operates at a given tier in the multi-tier framework, which includes templates replicated at each tier as an incumbent template. As noted, each template includes at least one of a plurality of policy elements. In general and as noted herein, a policy element can include, but is not limited to, a symptom surveillance regimen, a wellness surveillance regimen, a vitals surveillance regimen, a medication surveillance regimen, a procedure surveillance regimen, a goal surveillance pseudo regimen, and a file/attachment pseudo regimen. A plurality of such regimes can be implicated, and each regimen can include one or more values for definitions, thresholds, compliance rules, assessments, types, ranges, schedules, steps, etc.

At 1201 and 1202, the given tier receives an updated template of an incumbent template present at the given tier, and the updated template is parsed to determine included ones of the policy elements. In this example of FIG. 12, details for a symptom assessment regime, a vital/wellness regime, a medication regime, and procedure regime are shown. The example in FIG. 13 shows details for a goal pseudo regime and a file/attachment regime.

For each determined included policy element as generally shown in FIGS. 12-13, a determination is made if a value of the policy element in the updated template has been modified from a value previously provided in an earlier template stored as the incumbent template. For each determined included policy element, the value of the policy element in the updated template is placed as the value in the incumbent template when the value of the policy element has not been modified. Meanwhile, for each determined included policy element, when the value of the policy element in the updated template has been modified, the present value of the policy element is maintained in the incumbent template. In the end, the resultant incumbent template can be provided to a lower tier as an updated template.

Referring to item 1206, for example, there is shown process and logic for reconciling each symptom assessment regimen in a given version of a policy template at a given tier. With attention to 1208 and 1218, for each symptom, each applicable threshold in the incumbent policy template version is evaluated with respect to an updated version. As part of the evaluation, there is a determination as to whether the relevant field (e.g., value) in the incumbent policy template version has been modified or is protected by the participant owner of the incumbent version (or by another authorized source in some embodiments). In many embodiments, the modification and protection status of the data may be determined by checking a flag or indicator bit associated with the data, for example in a database or data structure. At 1228 a determination regarding modification or protection is made. If there has been no modification or protection, the update being propagated is made to the incumbent version 1241, and a new version ID may be assigned accordingly. It should be understood that some embodiments will apply either a modified or a protected determination and not both. Other embodiments may consider more specific metadata in the determination regarding whether the edits should propagate into a particular policy template, for example the identity of the participant-user, or organization proposing the edits may be used in combination with the modified and protected information.

Referring to item 1209, there is shown further process and logic for reconciling each symptom assessment regimen in a given version of a policy template. With attention to 1209 and 1219, for each schedule, the incumbent policy template version is evaluated with respect to the proposed updated version. As part of the evaluation, there is a determination as to whether the relevant field (e.g., value) in the incumbent policy template version has been modified or protected by the participant owner of the incumbent version (or by another authorized source in some embodiments). In many embodiments, the modification and protection status of the data may be determined by checking a flag or indicator bit associated with the data, for example in a database or data structure. At 1219 a determination regarding modification or protection is made and if there has been no modification or protection, the update being proposed and propagated is made to the incumbent version 1229, and a new version ID may be assigned accordingly. It should be understood that some embodiments will apply either a modified or a protected determination and not both. Other embodiments may consider more specific metadata in the determination regarding whether the edits should propagate into a particular policy template, for example the identity of the participant-user, or organization proposing the edits may be used in combination with the modified and protected information.

Referring to another example in item 1205, there is shown a process and logic for reconciling each vitals/wellness regimen in a given version of a policy template. As has been discussed above, vitals and wellness represent two different regimen types with practical similarities, which allow for their description to use a common illustration. The following illustrative example applies to both reconciliation of a wellness regimen and a reconciliation of a vitals regimen. With attention to 1210 and 1220, for each threshold, the incumbent policy template version is evaluated with respect to the proposed updated version. As part of the evaluation, there is a determination as to whether the relevant field in the incumbent policy template version has been modified or protected by the participant owner of the incumbent version, or in some embodiments by another authorized source. In many embodiments, the modification and protection status of the data may be determined by checking a flag or indicator bit associated with the data, for example in a database or data structure. At 1220 a determination regarding modification or protection is made and if there has been no modification or protection, the update being proposed and propagated is made to the incumbent version 1230, and a new version ID may be assigned accordingly. It should be understood that some embodiments will apply either a modified or a protected determination and not both. Other embodiments may consider more specific metadata in the determination regarding whether the edits should propagate into a particular policy template, for example the identity of the participant-user, or organization proposing the edits may be used in combination with the modified and protected information.

Referring now to item 1211a, there is shown further process and logic for reconciling each vitals and/or wellness regimen in a given version of a policy template. With attention to 1211a and 1221a, for each schedule, the incumbent policy template version is evaluated with respect to the proposed updated version. As part of the evaluation, there is a determination as to whether the relevant field in the incumbent policy template version has been modified or protected by the participant owner of the incumbent version, or in some embodiments by another authorized source. In many embodiments, the modification and protection status of the data may be determined by checking a flag or indicator bit associated with the data, for example in a database or data structure. At 1221a, a determination regarding modification or protection is made and if there has been no modification or protection, the update being proposed and propagated is made to the incumbent version 1231a, and a new version ID may be assigned accordingly. It should be understood that some embodiments will apply either a modified or a protected determination and not both. Other embodiments may consider more specific metadata in the determination regarding whether the edits should propagate into a particular policy template, for example the identity of the participant-user, or organization proposing the edits may be used in combination with the modified and protected information.

Referring now to item 1211b, there is shown further process and logic for reconciling each vitals and/or wellness regimen in a given version of a policy template. With attention to 1221b and 1231b, for each compliance rule, the incumbent policy template version is evaluated with respect to the proposed updated version. As part of the evaluation, there is a determination as to whether the relevant field in the incumbent policy template version has been modified or protected by the participant owner of the incumbent version, or in some embodiments by another authorized source. In many embodiments, the modification and protection status of the data may be determined by checking a flag or indicator bit associated with the data, for example in a database or data structure. At 1221b, a determination regarding modification or protection is made and if there has been no modification or protection, the update being proposed and propagated is made to the incumbent version 1231b, and a new version ID may be assigned accordingly. It should be understood that some embodiments will apply either a modified or a protected determination and not both. Other embodiments may consider more specific metadata in the determination regarding whether the edits should propagate into a particular policy template, for example the identity of the participant-user, or organization proposing the edits may be used in combination with the modified and protected information.

Referring now to yet another example at item 1207, there is shown a process and logic for reconciling each medication regimen in a given version of a policy template. With attention to 1212 and 1222, for each dosage, the incumbent policy template version is evaluated with respect to the proposed updated version. As part of the evaluation, there is a determination as to whether the relevant field in the incumbent policy template version has been modified or protected by the participant owner of the incumbent version, or in some embodiments by another authorized source. In many embodiments, the modification and protection status of the data may be determined by checking a flag or indicator bit associated with the data, for example in a database or data structure. At 1222 a determination regarding modification or protection is made and if there has been no modification or protection, the update being proposed and propagated is made to the incumbent version 1232, and a new version ID may be assigned accordingly. It should be understood that some embodiments will apply either a modified or a protected determination and not both. Other embodiments may consider more specific metadata in the determination regarding whether the edits should propagate into a particular policy template, for example the identity of the participant-user, or organization proposing the edits may be used in combination with the modified and protected information.

Referring now to item 1213, there is shown further process and logic for reconciling each medication regimen in a given version of a policy template. With attention to 1213 and 1223, for each titration, the incumbent policy template version is evaluated with respect to the proposed updated version. As part of the evaluation, there is a determination as to whether the relevant field in the incumbent policy template version has been modified or protected by the participant owner of the incumbent version, or in some embodiments by another authorized source. In many embodiments, the modification and protection status of the data may be determined by checking a flag or indicator bit associated with the data, for example in a database or data structure. At 1223 a determination regarding modification or protection is made and if there has been no modification or protection, the update being proposed and propagated is made to the incumbent version 1233, and a new version ID may be assigned accordingly. It should be understood that some embodiments will apply either a modified or a protected determination and not both. Other embodiments may consider more specific metadata in the determination regarding whether the edits should propagate into a particular policy template, for example the identity of the participant-user, or organization proposing the edits may be used in combination with the modified and protected information.

Referring now to item 1214, there is shown further process and logic for reconciling each medication regimen in a given version of a policy template. With attention to 1214 and 1224, for each medication or substituted medication, the incumbent policy template version is evaluated with respect to the proposed updated version. As part of the evaluation, there is a determination as to whether the relevant field in the incumbent policy template version has been modified or protected by the participant owner of the incumbent version, or in some embodiments by another authorized source. In many embodiments, the modification and protection status of the data may be determined by checking a flag or indicator bit associated with the data, for example in a database or data structure. At 1224 a determination regarding modification or protection is made and if there has been no modification or protection, the update being proposed and propagated is made to the incumbent version 1234, and a new version ID may be assigned accordingly. It should be understood that some embodiments will apply either a modified or a protected determination and not both. Other embodiments may consider more specific metadata in the determination regarding whether the edits should propagate into a particular policy template, for example the identity of the participant-user, or organization proposing the edits may be used in combination with the modified and protected information.

Referring now to item 1215, there is shown further process and logic for reconciling each medication regimen in a given version of a policy template. With attention to 1215 and 1225, for each compliance rule, the incumbent policy template version is evaluated with respect to the proposed updated version. As part of the evaluation, there is a determination as to whether the relevant field in the incumbent policy template version has been modified or protected by the participant owner of the incumbent version, or in some embodiments by another authorized source. In many embodiments, the modification and protection status of the data may be determined by checking a flag or indicator bit associated with the data, for example in a database or data structure. At 1225 a determination regarding modification or protection is made and if there has been no modification or protection, the update being proposed and propagated is made to the incumbent version 1235, and a new version ID may be assigned accordingly. It should be understood that some embodiments will apply either a modified or a protected determination and not both. Other embodiments may consider more specific metadata in the determination regarding whether the edits should propagate into a particular policy template, for example the identity of the participant-user, or organization proposing the edits may be used in combination with the modified and protected information.

Referring now to a further example at item 1240, there is shown a process and logic for reconciling each procedure regimen in a given version of a policy template. With attention to 1216 and 1226, for each compliance rule, the incumbent policy template version is evaluated with respect to the proposed updated version. As part of the evaluation, there is a determination as to whether the relevant field in the incumbent policy template version has been modified or protected by the participant owner of the incumbent version, or in some embodiments by another authorized source. In many embodiments, the modification and protection status of the data may be determined by checking a flag or indicator bit associated with the data, for example in a database or data structure. At 1226 a determination regarding modification or protection is made and if there has been no modification or protection, the update being proposed and propagated is made to the incumbent version 1236, and a new version ID may be assigned accordingly. It should be understood that some embodiments will apply either a modified or a protected determination and not both. Other embodiments may consider more specific metadata in the determination regarding whether the edits should propagate into a particular policy template, for example the identity of the participant-user, or organization proposing the edits may be used in combination with the modified and protected information.

Referring now to item 1217, there is shown further process and logic for reconciling each procedure regimen in a given version of a policy template. With attention to 1217 and 1227, for each schedule, the incumbent policy template version is evaluated with respect to the proposed updated version. As part of the evaluation, there is a determination as to whether the relevant field in the incumbent policy template version has been modified or protected by the participant owner of the incumbent version, or in some embodiments by another authorized source. In many embodiments, the modification and protection status of the data may be determined by checking a flag or indicator bit associated with the data, for example in a database or data structure. At 1227 a determination regarding modification or protection is made and if there has been no modification or protection, the update being proposed and propagated is made to the incumbent version 1237, and a new version ID may be assigned accordingly. It should be understood that some embodiments will apply either a modified or a protected determination and not both. Other embodiments may consider more specific metadata in the determination regarding whether the edits should propagate into a particular policy template, for example the identity of the participant-user, or organization proposing the edits may be used in combination with the modified and protected information.

In the examples provided above, a “field” has been described as the protected element. The examples do not exhaustively show every possible field of interest. As one skilled in the art will appreciate based on the disclosure, however, the examples illustrate the alteration generically at a field level, which can be used for any given field.

### X. Reconciliation of Goals and File/Attachment Regimens

Conceptually, embodiments of the present disclosure contemplate reconciliation of edits/customizations from any type of regimen so that editing may propagate to any participant or tier in the system. In addition, embodiments envision using the reconciliation logic described above to reconcile regimens or pseudo regimens of many kinds, including goals and file/attachment regimens.

Referring now to item 1307, there is shown a process and logic for reconciling each goals pseudo-regimen in a given version of a policy template. With attention to 1312 and 1322, for each goal name, the incumbent policy template version is evaluated with respect to the proposed updated version. As part of the evaluation, there is a determination as to whether the relevant field in the incumbent policy template version has been modified or protected by the participant owner of the incumbent version, or in some embodiments by another authorized source. In many embodiments, the modification and protection status of the data may be determined by checking a flag or indicator bit associated with the data, for example in a database or data structure. At 1322 a determination regarding modification or protection is made and if there has been no modification or protection, the update being proposed and propagated is made to the incumbent version 1332, and a new version ID may be assigned accordingly. It should be understood that some embodiments will apply either a modified or a protected determination and not both. Other embodiments may consider more specific metadata in the determination regarding whether the edits should propagate into a particular policy template, for example the identity of the participant-user, or organization proposing the edits may be used in combination with the modified and protected information.

Referring now to item 1313, there is shown further process and logic for reconciling each goal regimen in a given version of a policy template. With attention to 1313 and 1323, for each goal definition, the incumbent policy template version is evaluated with respect to the proposed updated version. As part of the evaluation, there is a determination as to whether the relevant field in the incumbent policy template version has been modified or protected by the participant owner of the incumbent version, or in some embodiments by another authorized source. In many embodiments, the modification and protection status of the data may be determined by checking a flag or indicator bit associated with the data, for example in a database or data structure. At 1323 a determination regarding modification or protection is made and if there has been no modification or protection, the update being proposed and propagated is made to the incumbent version 1333, and a new version ID may be assigned accordingly. It should be understood that some embodiments will apply either a modified or a protected determination and not both. Other embodiments may consider more specific metadata in the determination regarding whether the edits should propagate into a particular policy template, for example the identity of the participant-user, or organization proposing the edits may be used in combination with the modified and protected information.

Referring now to item 1314, there is shown further process and logic for reconciling each goal regimen in a given version of a policy template. With attention to 1314 and 1324, for each compliance rule, the incumbent policy template version is evaluated with respect to the proposed updated version. As part of the evaluation, there is a determination as to whether the relevant field in the incumbent policy template version has been modified or protected by the participant owner of the incumbent version, or in some embodiments by another authorized source. In many embodiments, the modification and protection status of the data may be determined by checking a flag or indicator bit associated with the data, for example in a database or data structure. At 1324 a determination regarding modification or protection is made and if there has been no modification or protection, the update being proposed and propagated is made to the incumbent version 1334, and a new version ID may be assigned accordingly. It should be understood that some embodiments will apply either a modified or a protected determination and not both. Other embodiments may consider more specific metadata in the determination regarding whether the edits should propagate into a particular policy template, for example the identity of the participant-user, or organization proposing the edits may be used in combination with the modified and protected information.

Referring now to item 1340, there is shown a process and logic for reconciling each file/attach regimen in a given version of a policy template. With attention to 1316 and 1326, for each compliance rule, the incumbent policy template version is evaluated with respect to the proposed updated version. As part of the evaluation, there is a determination as to whether the relevant field in the incumbent policy template version has been modified or protected by the participant owner of the incumbent version, or in some embodiments by another authorized source. In many embodiments, the modification and protection status of the data may be determined by checking a flag or indicator bit associated with the data, for example in a database or data structure. The evaluation at Step 1316 refers to revising a file/attachment within the allowed file types. Additionally, a list of allowed file types can also be changed and propagated down.

At 1326 a determination regarding modification or protection is made and if there has been no modification or protection, the update being proposed and propagated is made to the incumbent version 1336, and a new version ID may be assigned accordingly. It should be understood that some embodiments will apply either a modified or a protected determination and not both. Other embodiments may consider more specific metadata in the determination regarding whether the edits should propagate into a particular policy template, for example the identity of the participant-user, or organization proposing the edits may be used in combination with the modified and protected information.

Referring now to item 1317, there is shown further process and logic for reconciling each file/attachment regimen in a given version of a policy template. With attention to 1317 and 1327, for each schedule, the incumbent policy template version is evaluated with respect to the proposed updated version. As part of the evaluation, there is a determination as to whether the relevant field in the incumbent policy template version has been modified or protected by the participant owner of the incumbent version, or in some embodiments by another authorized source. In many embodiments, the modification and protection status of the data may be determined by checking a flag or indicator bit associated with the data, for example in a database or data structure. At 1327 a determination regarding modification or protection is made and if there has been no modification or protection, the update being proposed and propagated is made to the incumbent version 1337, and a new version ID may be assigned accordingly. It should be understood that some embodiments will apply either a modified or a protected determination and not both. Other embodiments may consider more specific metadata in the determination regarding whether the edits should propagate into a particular policy template, for example the identity of the participant-user, or organization proposing the edits may be used in combination with the modified and protected information.

Finally, items 1339 and 1304 reflect the repetitive application of the reconciliation logic 1301 to subscribed, prescribed, or used policy templates moving sequentially down (or in some embodiments up) tiers of participants.

Not shown for space reasons is the flow from any determination of modified or protected state, such as 1219-1228 and 1322-1327, if the determination indicates that the value is modified or protected. In that “yes” instance, flow proceeds to item 1338 which indicates the inclusion on all completed activities in the process, bypassing the relevant change operation.

Finally, items 1339 and 1204 reflect the repetitive application of the reconciliation logic 1201 to subscribed, prescribed, or used policy templates moving sequentially down (or in some embodiments up) tiers of participants.

### Y. Reconciliation of White Space and Other Items

As shown in FIG. 5B, embodiments of the disclosure illustrate and contemplate white space regimens with minimal structure, so diagrammatic reconciliation processes are not as illustrative as the examples above where structure can be used as a mechanism for conveying principles. There are, however, embodiments of the disclosure that contemplate reconciling functionality for any policy template within the scope of this disclosure and perhaps beyond. The underlying principles for this reconciling are as follows: Any regimen aspect that is created, may have a process for reconciling, so the diagrams above are not intended to illustrate all of the possibilities for data reconciliation, even for the disclosed regimen types; any edits/modifications made should have capability for reconciliations in other versions of the subject template, or even other templates where possible; reconciliation may consider the history of editing to a template as part of the determination regarding whether to apply a particular edit to a particular template; if a field has been modified prior to the reconciliation, other factors such as the source of the edits (entity or individual) may be considered in the determination of whether to apply the edit; if a field has been protected by another participant prior to the reconciliation, other factors such as the source of the edits (entity or individual) may be considered in the determination of whether to apply the edit; participants may be provided options for how edits should propagate to templates they are using, prescribing or publishing, and those preferences can be specific to each field and each downstream participant; and, the combination of field protection and selection of what fields are subject to reconciliation can form the basis for a no-conflict system or a limited conflict system.

FIGS. 12 and 13 illustrate template changes being promulgated in a push model, where any changed templates are provided to subscribing participants at the next lower tier automatically upon completion of the template changes. In some embodiments, template changes can also be promulgated in a pull model, where each participant device periodically checks either the centralized server 403 or the server of the next higher tier to determine if a changed template is available. If a changed template is available, the changed template is retrieved and the process of FIGS. 12 and 13 is performed on the changed template. Thus, items 1204 and 1339 are performed periodically, rather than directly after completion of operation at a given tier. In some embodiments, a combination of push and pull models is used, the pull portion being used in case a given participant device is not available when the operation at the next higher level completes to receive the pushed changed template.

While the operations of FIGS. 12 and 13 are focused on changes to existing elements in a template, it is understood that if the template includes a new policy element, that is considered a change, and the policy element is added to the template.

While the operations of FIGS. 12 and 13 describe changing the value of a regimen element if not modified or protected, it is understood that an additional determination can be made on whether the value of the regimen element is different between the updated template and the incumbent template. If the values are the same, the storing of the value in the updated template into the incumbent template can be avoided, saving numerous write operations in most cases. The order of this changed value determination and the modified or protected determination is not specific and can be performed in either order. In some examples, performing the changed value determination can be performed first and then the modified or protected determination, as it is likely that more values in a template will be the same than are modified or protected.

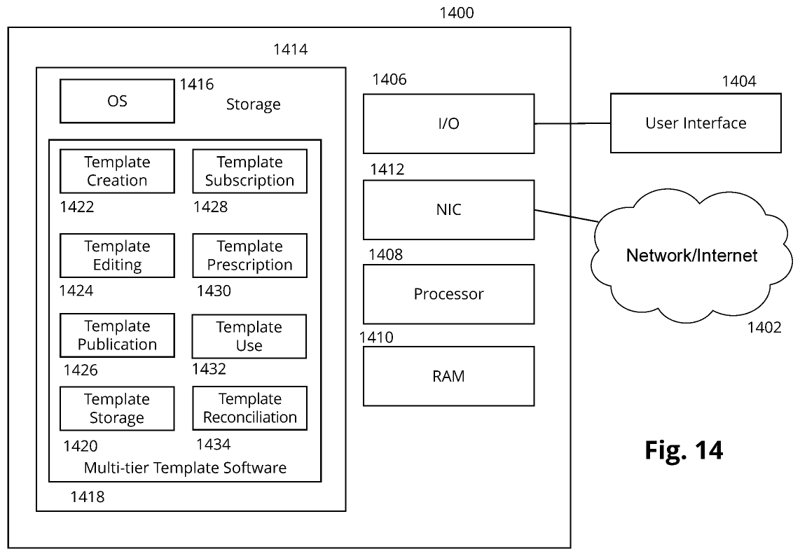

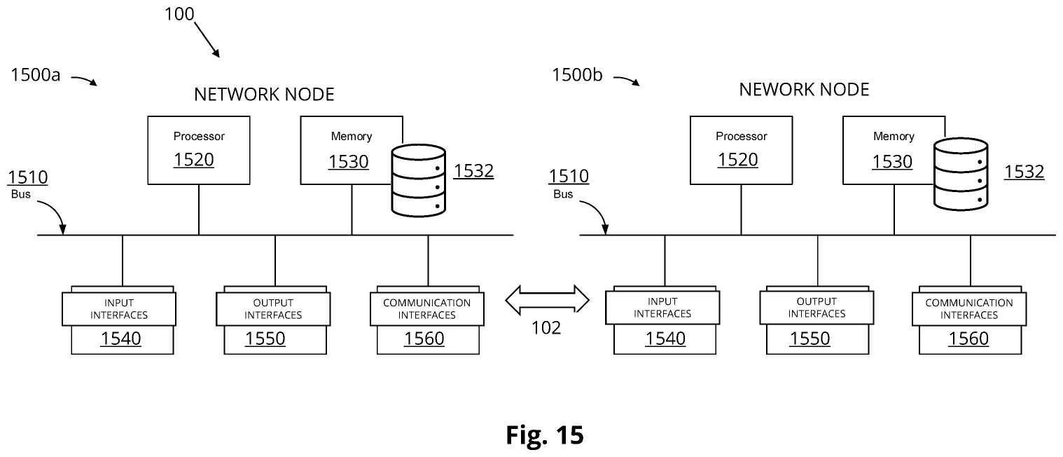

FIG. 14 illustrates an exemplary computer 1400 for performing the operations described above. The computer 1400 is connected to a network and/or the Internet 1402, such as network 102. A user interface 1404 is connected to an I/O module 1406 inside the computer 1400. This allows users of the computer 1400 to provide inputs to the process and to receive results of the process.

The computer 1400 includes a processor 1408, RAM 1410 used to store programs and data during operation of the system 300, a network interface card (NIC) 142 to connect to the network 1402, and non-volatile program storage 1414. Programs contained in the storage 1414 are an operating system 1416 and an instance of the multi-tier template software 1418, which is the software providing the logic for the above operations and processes. Template storage 1420 is provided to store the various templates present in the computer 1400. Software modules include template creation 1422, template editing 1424, template publication 1426, template subscription 1428, template prescription 1430, template use 1432 and template reconciliation 1434. The template use module 1432 may have two variations, one for patient use and for prescriber use, to monitor the patient use and results. In many embodiments, patient tier software will include only the template subscription 1428, template use 1432 and template reconciliation 1434 modules, as it is not desirable to allow a patient to create, edit, publish, or prescribe templates. In many embodiments, the tiers above the patient tier or prescriber tier may not include the template use 1432 module. All tiers except the top tier would include the template reconciliation 1434 module operating as shown in FIGS. 12 and 13 to allow flow down of templates without removing any modifications and protected policies.

It is understood that this is a highly simplified illustration of the computer 1400 and an actual computer may be configured in numerous different ways to perform the operations. With a “serverless” distributed system, the logic the multi-tier template software disclosed herein can also be distributed among many computers that work in concert to achieve the functional objects described in this disclosure. For example, FIG. 15 schematically illustrates components of network devices or nodes 300a-b of the disclosed network system 100 in one or more networks (102) of FIG. 1. As shown in FIG. 15, each network node 1500a-b may include a bus 1510, a processor 1520, a memory 1530, and a communication interface 1540. Other components, such as an input and output interface 1540, 1550, may be available.

The bus 1510 includes one or more components that enable wired and/or wireless communication among the components of the network node 1500a-b. The bus 1510 may couple together two or more components of FIG. 15, such as via operative coupling, communicative coupling, electronic coupling, and/or electric coupling. The processor 1520 includes a central processing unit, a microprocessor, a controller, a microcontroller, a digital signal processor, a field-programmable gate array, an application-specific integrated circuit, and/or another type of processing component. The processor 1520 is implemented in hardware, firmware, or a combination of hardware and software. In some implementations, the processor 1520 includes one or more processors capable of being programmed to perform one or more operations or processes described elsewhere herein.

The memory 1530 includes volatile and/or nonvolatile memory. For example, the memory 1530 may include random access memory (RAM), read only memory (ROM), a hard disk drive, and/or another type of memory (e.g., a flash memory, a magnetic memory, and/or an optical memory). The memory 1530 may include internal memory (e.g., RAM, ROM, or a hard disk drive) and/or removable memory (e.g., removable via a universal serial bus connection). The memory 1530 may be a non-transitory computer-readable medium. The memory 1530 stores information, instructions, and/or software (e.g., one or more software applications) related to the operation of the network node 1500a-b. In some implementations, the memory 1530 includes one or more memories that are coupled to one or more processors (e.g., the processor 1520), such as via the bus 1510.

For at least one network node 1500a, the input/output interfaces 1540, 1550 enables the network node 1500a to receive input, such as user input and/or sensed input. For example, the input/output interfaces 1540, 1550 may include a touch screen, a keyboard, a keypad, a mouse, a button, a microphone, a switch, a sensor, etc. The input/output interfaces 1540, 1550 also enable the network node 1500a to provide output, such as via a display, a speaker, etc.

The communication interface 1540 enables the network node 1500a-b to communicate with other devices via a wired connection and/or a wireless connection. For example, the communication interface 1540 may include a receiver, a transmitter, a transceiver, a modem, a network interface card, and/or an antenna.

The network node 1500a-b may perform one or more operations or processes described herein. For example, a non-transitory computer-readable medium (e.g., the memory 1530) may store a set of instructions (e.g., one or more instructions or code) for execution by the processor 1520. The processor 1520 may execute the set of instructions to perform one or more operations or processes described herein. In some implementations, execution of the set of instructions, by one or more processors 1520, causes the one or more processors 1520 and/or the network node 1500a-b to perform one or more operations or processes described herein. In some implementations, hardwired circuitry may be used instead of or in combination with the instructions to perform one or more operations or processes described herein. Additionally, or alternatively, the processor 1520 may be configured to perform one or more operations or processes described herein. Thus, implementations described herein are not limited to any specific combination of hardware circuitry and software.

As disclosed herein, the system (e.g., distributed network system) 100 operates within one or more networks of a decentralized health information network 102, connecting with numerous network nodes, which are associated with patients, providers, and payers, to facilitate the purposes of the present disclosure. The several components for the disclosed system 100 can be incorporated into one or more of the network node 1500a-b, can be shared between one or more of the network node 1500a-b, and/or can be distributed among several of the network node 1500a-b. (Although two network node 1500a-b are shown, it will be appreciated that the network system 100 can include any plurality of such devices.) Therefore, the disclosed system 100 generally includes one or more databases 1532 (e.g., in memory 1530), one or more interfaces (e.g., 1540, 1550, 1560), and one or more processors (e.g., processors 1520) incorporated into one or more of the network node 1500a-b, shared between several of the network node 1500a-b, and/or distributed among several of the network node 1500a-b.

The one or more databases 1532 in memory 1530 store information to achieve the purposes of the present disclosure. The one or more processors 1520 are in operational communication with the databases 1532 and the interfaces 1540, 1550, 1560. These processors 1520 are configured to perform the purposes of the present disclosure. Additionally, the processors 1520 can build a distributed ledger for a given patient (20), and the processors 1520 can be configured to distribute the distributed ledger within the health information network 102.

The number and arrangement of components shown in FIG. 15 are provided as an example. The network node 1500a-b may include additional components, fewer components, different components, or differently arranged components than those shown in FIG. 15. Additionally, or alternatively, a set of components (e.g., one or more components) of the network node 1500a-b may perform one or more functions described as being performed by another set of components of the network node 1500a-b.

The techniques of the present disclosure can be implemented in digital electronic circuitry, or in computer hardware, firmware, software, or in combinations of these. Apparatus for practicing the disclosed techniques can be implemented in a computer program product tangibly embodied in a machine-readable storage device for execution by a programmable processor; and method steps of the disclosed techniques can be performed by a programmable processor executing a program of instructions to perform functions of the disclosed techniques by operating on input data and generating output. The disclosed techniques can be implemented advantageously in one or more computer programs that are executable on a programmable system including at least one programmable processor coupled to receive data and instructions from, and to transmit data and instructions to, a data storage system, at least one input device, and at least one output device. Each computer program can be implemented in a high-level procedural or object-oriented programming language, or in assembly or machine language if desired; and in any case, the language can be a compiled or interpreted language. Suitable processors include, by way of example, both general and special purpose microprocessors. Generally, a processor will receive instructions and data from a read-only memory and/or a random-access memory. Generally, a computer will include one or more mass storage devices for storing data files; such devices include magnetic disks, such as internal hard disks and removable disks; magneto-optical disks; and optical disks. Storage devices suitable for tangibly embodying computer program instructions and data include all forms of non-volatile memory, including by way of example semiconductor memory devices, such as EPROM, EEPROM, and flash memory devices; magnetic disks such as internal hard disks and removable disks; magneto-optical disks; and CD-ROM disks. Any of the foregoing can be supplemented by, or incorporated in, ASICs (application-specific integrated circuits).

While the above description has focused on patient care, it is understood that the process can be applied to many different types of multi-tier environments, where templates are provided at a higher level and transferred down through multiple tiers. The evaluation of each template policy element as new or updated templates is provided to each tier follows the process of FIGS. 12 and 13, with just the specifics of the actual meaning of the values changing.

One feature of embodiments of the present disclosure is that the software can be essentially identical at each tier in a multi-tier arrangement, particularly for all but the patient or lowest tier. This simplifies maintenance of the software. By including the reconciliation operation as described in all tiers, except the top tier, modifications and protections can be maintained at all lower tiers when a higher tier publishes an updated template. The need to redo modifications after each update is removed, simplifying overall system operation, and increasing the likelihood of use of the multi-tier system, with the resulting improvements in patient care and wellbeing.

It is to be understood that the above description is intended to be illustrative, and not restrictive. The material has been presented to enable any person skilled in the art to make and use the disclosed subject matter as claimed and is provided in the context of particular embodiments, variations of which will be readily apparent to those skilled in the art (e.g., many of the disclosed embodiments may be used in combination with each other). In addition, it will be understood that some of the operations identified herein may be performed in different orders. The scope of the disclosed subject matter, therefore, should be determined with reference to the appended claims, along with the full scope of equivalents to which such claims are entitled. In the appended claims, the terms “including” and “in which” are used as the plain-English equivalents of the respective terms “comprising” and “wherein.” Finally, various modifications and changes can be made to the principles and examples described herein without departing from the scope of the disclosure and without departing from the claims which follow.

1. A method of operating a given tier in a multi-tier framework, the multi-tier framework including templates replicated at each tier as an incumbent template, each template including at least one of a plurality of policy elements, the method comprising:
receiving at the given tier an updated template of an incumbent template present at the given tier;
parsing the updated template to determine included ones of the policy elements;
for each determined included policy element, determining if a value of the policy element in the updated template has been modified from a value previously provided in an earlier template stored as the incumbent template;
for each determined included policy element, when the value of the policy element has not been modified, placing the value of the policy element in the updated template as the value in the incumbent template;
for each determined included policy element, when the value of the policy element in the updated template has been modified, maintaining the present value of the policy element in the incumbent template; and
providing the resultant incumbent template to a lower tier as an updated template.

2. The method of claim 1, wherein the one or more policy elements are selected from the group consisting of: a symptom surveillance regimen, a wellness surveillance regimen, a vitals surveillance regimen, a medication surveillance regimen, and a procedure surveillance regimen.

3. The method of claim 2, wherein the group for the one or more policy elements further consists of: a goal surveillance pseudo regimen and a file/attachment pseudo regimen.

4. The method of claim 1, wherein the one or more policy elements comprise a plurality of system surveillance regimes; and wherein each symptom surveillance regimen comprises one or more values of: one or more symptom definitions, one or more thresholds associated with the symptom definitions, one or more compliance rules associated with the symptom definitions, and one or more schedules assessments associated with the symptom definitions.

5. The method of claim 1, wherein the one or more policy elements comprise a plurality of wellness surveillance regimes; and wherein each wellness surveillance regimen comprises one or more values of: a wellness type, one or more ranges associated with the wellness type, one or more thresholds associated with the wellness type, one or more compliance rules associated with the wellness type, and one or more schedule assessments associate with the wellness type.

6. The method of claim 1, wherein the one or more policy elements comprise a plurality of vitals surveillance regimes; and wherein each vitals surveillance regimen comprises one or more values of: a vitals type, one or more ranges associated with the vitals type, one or more thresholds associated with the vitals type, one or more compliance rules associated with the vitals type, and one or more schedule assessments associate with the vitals type.

7. The method of claim 1, wherein the one or more policy elements comprise a plurality of medication surveillance regimes; and wherein each medication surveillance regimen comprises one or more values of: an identity of a medication, one or more dosages associated with the identity of medication, one or more compliance rules associated with the identity of medication, and one or more schedules associated with the identity of medication, wherein the schedules associated with the identity of medication include titration information.

8. The method of claim 1, wherein the one or more policy elements comprise a plurality of procedure surveillance regimes; and wherein each procedure surveillance regimen comprises one or more values of: a definition of procedural steps, one or more compliance rules associated with the definition of procedural steps, and one or more schedules associated with the definition of procedural steps.

9. The method of claim 1, wherein to receive at the given tier the updated template of the incumbent template present at the given tier comprises subscribing, at the given tier, to a higher tier providing the updated template.

10. The method of claim 1, wherein providing the resultant incumbent template to the lower tier as the updated template comprises providing the resultant incumbent template to the lower tier that subscribes to the given tier.

11. The method of claim 1, wherein the method further comprises receiving a request to modify a given one of the policy elements of the incumbent template at the given tier.

12. The method of claim 11, wherein, in response to the request to modify the given policy element of the incumbent template at the given tier, the method comprises:
determining if the given policy element reflects a prior modification; and
accepting or applying any new modification based upon that determination.

13. The method of claim 11, wherein, in response to the request to modify the given policy element of the incumbent template at the given tier, the method comprises:
determining that the given policy element is indicated as being protected from modification; and
denying any new modification based upon that determination.

14. The method of claim 1, further comprising, for each determined included policy element, when the value of the policy element is indicated as being protected, maintaining the present value of the policy element in the incumbent template.

15. The method of claim 1, wherein the determined included policy element is a symptom surveillance regimen; and wherein determining if the value of the policy element has been modified from the value previously provided in the earlier template stored as the incumbent template comprises:
determining if the value of one or more of a threshold, a schedule, and a compliance rule associated with the symptom surveillance regimen reflects a prior modification, and placing the value or maintaining the present value based on that determination; or
determining if a new symptom surveillance regime has been added to the updated template, and placing the value of one or more of a name for the new symptom surveillance regime, a symptom definition, an associated threshold, an associated compliance rule, and an associated schedule assessment in the incumbent template.

16. The method of claim 1, wherein the determined included policy element is a wellness surveillance regimen; and wherein determining if the value of the policy element has been modified from the value previously provided in the earlier template stored as the incumbent template comprises:
determining if the value of one or more of a range, a threshold, a regime schedule, and a compliance rule associated with the wellness surveillance regimen reflects a prior modification, and placing the value or maintaining the present value based on that determination; or
determining if a new wellness regimen has been added to the updated template, and placing the value of one or more of a wellness type, an acceptable range, one or more thresholds, one or more compliance rules, and a schedule assessment associated with the new wellness regimen in the incumbent template.

17. The method of claim 1, wherein the determined included policy element is a vitals surveillance regimen; and wherein determining if the value of the policy element has been modified from the value previously provided in the earlier template stored as the incumbent template comprises:
determining if the value of one or more of a range, a threshold, a regime schedule, and a compliance rule associated with the vitals surveillance regimen reflects a prior modification, and placing the value or maintaining the present value based on that determination; or
determining if a new vitals regimen has been added to the updated template, and placing the value of one or more of a vitals type, an acceptable range, one or more thresholds, one or more compliance rules, and a schedule assessment associated with the new vitals regimen in the incumbent template.

18. The method of claim 1, wherein the determined included policy element is a medication surveillance regimen; and wherein determining if the value of the policy element has been modified from the value previously provided in the earlier template stored as the incumbent template comprises:
determining if the value of one or more of a substitute medication, a dosage, a titration schedule, and a compliance rule associated with the medication surveillance regimen reflects a prior modification, and placing the value or maintaining the present value based on that determination; or
determining if a new medication regimen has been added to the updated template, and placing the value of one or more of a medication identity, a dosage, one or more compliance rules, and a titration schedule associated with the new medication regimen in the incumbent template.

19. The method of claim 1, wherein the determined included policy element is a procedure surveillance regimen; and wherein determining if the value of the policy element has been modified from values previously provided in the earlier template stored as the incumbent template comprises:
determining if the value of one or more of a regimen schedule medication and a compliance rule associated with the procedure surveillance regimen reflects a prior modification, and placing the value or maintaining the present value based on that determination; or
determining if a new procedure regimen has been added to the updated template, and placing the value of one or more of a procedural step, a compliance rule, and a schedule assessment in the incumbent template.

20. The method of claim 1, wherein determining if the value of the policy element has been modified from the value previously provided in the earlier template stored as the incumbent template comprises:
determining that the updated template reflects a prior modification for one or more aspects of the policy element; and
denying any new modification of the incumbent template for the respective aspect where the prior modification is found, thereby maintaining the present value of the policy element in the incumbent template.

21. The method of claim 1, wherein determining if the value of the policy element has been modified from the value previously provided in the earlier template stored as the incumbent template comprises:
determining that the updated template reflects a prior modification for one or more aspects of the policy element; and
allowing only a new modification of the updated template for the respective aspect when the prior modification is found if the modification for the new modification is proposed by a same participant that made the prior modification, thereby placing the value in the updated template as the value in the incumbent template.

22. The method of claim 1, wherein determining if the value of the policy element has been modified from the value previously provided in the earlier template stored as the incumbent template comprises:
determining if the updated template reflects a prior modification for one or more aspects of a threshold, a regimen schedule, a compliance rule, a range, a medication, a dosage, or a titration schedule;
denying any new modification of the updated template for the respective aspect where a prior modification is found, thereby maintaining the present value of the policy element in the incumbent template; and
allowing only a new modification of the updated template for the respective aspect when the prior modification is found if the modification for the new modification is proposed by a same participant that made the prior modification, thereby placing the value in the updated template as the value in the incumbent template.

23. The method of claim 1, wherein the method further comprises:
receiving a request to create a template from a creator-participant; and
permitting the creator-participant to designate one or more aspects of the template as protected.

24. The method of claim 23, wherein the designation as protected comprises at least one of: preventing any modification of the aspect; preventing any modification of the aspect by a participant or a participant type designated by the creator-participant; and preventing any modification of the aspect by a participant or a participant type not designated by the creator-participant.

25. The method of claim 23, wherein a modification of a template includes a capability of deleting a pre-existing regimen.

26. A programmable storage device having program instructions stored thereon for causing one or more programmable network devices to perform a method of claim 1.
